(19) United States
(12) Patent Application Publication
(10) Pub. No.: US 2004/0157878 A1
(43) Pub. Date: Aug. 12, 2004

(54) Compounds as radioligands for the diagnosis of disease

(76) Inventors: Bruce Nelson Rogers, Mystic, CT (US); David W. Piotrowski, Groton Long Point, CT (US); Vincent Edward Groppi JR., Kalamazoo, MI (US); Eric Jon Jacobsen, Chesterfield, MO (US); Jason Kenneth Myers, Indianapolis, IN (US); Daniel Patrick Walker, Noank, CT (US); Donn Gregory Wishka, Groton Long Point, CT (US); Marc Bradley Skaddan, Richland, MI (US)

Correspondence Address: PHARMACIA & UPJOHN, 301 HENRIETTA ST, 0228-32-LAW, KALAMAZOO, MI, 49007, US

(21) Appl. No.: 10/729,529

(22) Filed: Dec. 5, 2003

Related U.S. Application Data
(60) Provisional application No. 60/431,473, filed on Dec. 6, 2002.

(51) Int. Cl. C07D 471/02; A61K 31/4745; A61K 31/46

(52) U.S. Cl. 514/303; 514/304; 514/305; 514/412; 546/118; 546/125; 546/135

(57) ABSTRACT

Radiolabeled ligands useful as probes for determining the relative abundance, receptor occupancy, and/or function of nicotinic acetylcholine receptors. The compounds of Formula I as described herein are labeled with a radioactive isotopic moiety such as ¹¹C, ¹⁸F, ⁷⁶Br, ¹²³I or ¹²⁵I. Disorders are diagnosed by administering to a mammal a detectably labeled compound and detecting the binding of that compound to the nAChR. The compounds that have been administered are detected using methods including, but not limited to, position emission topography and single-photon emission computed tomography. The present invention is useful in diagnosing a wide variety of diseases and disorders as discussed herein.

### CROSS REFERENCE TO RELATED APPLICATIONS

This application claims the benefit of U.S. provisional application Serial No. 60/431,473 filed on 6 Dec. 2002, under 35 USC 119(e)(i), which is incorporated herein by reference in its entirety.

### FIELD OF INVENTION

Nicotinic acetylcholine receptors (nAChRs) play a large role in central nervous system (CNS) activity. Particularly, they are known to be involved in cognition, learning, mood, emotion, and neuroprotection. There are several types of nicotinic acetylcholine receptors, and each one appears to have a different role in regulating CNS function. The present invention relates to molecules that have a greater effect upon the α7 nAChRs as compared to other closely related members of this large ligand-gated receptor family. Compounds of the present invention are radiolabeled alpha 7 agonists that are useful as imaging agents and biomarkers for medical therapy and diagnosis. Such radiolabeled compounds are also useful as pharmacological tools for studying nAChR function and activity. Accordingly, the invention also provides a radiolabeled compound of the present invention, or a salt thereof.

### BACKGROUND OF THE INVENTION

The α7 nAChR is one receptor system that has proved to be a difficult target for testing. Native α7 nAChR is not routinely able to be stably expressed in most mammalian cell lines (Cooper and Millar, J. Neurochem., 1997, 68(5):2140-51). Another feature that makes functional assays of α7 nAChR challenging is that the receptor is rapidly (100 milliseconds) inactivated. This rapid inactivation greatly limits the functional assays that can be used to measure channel activity.

Recently, Eisele et al. has indicated that a chimeric receptor formed between the N-terminal ligand binding domain of the α7 nAChR (Eisele et al., Nature, 366(6454), p 479-83, 1993), and the pore forming C-terminal domain of the 5-HT₃ receptor expressed well in Xenopus oocytes while retaining nicotinic agonist sensitivity. Eisele et al. used the N-terminus of the avian (chick) form of the α7 nAChR receptor and the C-terminus of the mouse form of the 5-HT₃ gene. However, under physiological conditions the α7 nAChR is a calcium channel while the 5-HT₃R is a sodium and potassium channel. Indeed, Eisele et al. teaches that the chicken α7 nAChR/mouse 5-HT₃R behaves quite differently than the native α7 nAChR with the pore element not conducting calcium but actually being blocked by calcium ions. WO 00/73431 A2 reports on assay conditions under which the 5-HT₃R can be made to conduct calcium. This assay may be used to screen for agonist activity at this receptor.

The distribution and function of nicotinic cholinergic receptors within the body is consistent with the view that nicotinic cholinergic signaling is involved in the regulation of the key neurochemicals in the brain and where other nAChRs are found and influence nicotine-sensitive neuronal processes involved in processes including sensory processing and cognition. Cholinergic neurons are located in a number of regions throughout the brain and other areas, and there are a number of neurotransmitters whose release is modulated by effects upon nicotinic cholinergic receptors. Certain nicotinic cholinergic receptor subtypes have been recognized as targets for diagnostic imaging. See, Villemagne et al., in: Arneric et al. (Eds.) Neuronal Nicotinic Receptors: Pharmacology and Therapeutic Opprotunities, 235-250 (1998). Furthermore, efforts have been directed toward development of radiotracers that image certain nicotinic receptors within the brain. See, Guan et al., J. Neurochem., 74(1):237-243 (2000).

There have been efforts to develop non-invasive techniques to probe neuro-receptors in vivo. Positron emission tomography (PET) and single photon emission computed tomography (SPECT) of high affinity ligands to map and monitor alterations in receptor densities for a variety of receptor targets having relevance to human disease has been investigated. For example, studies using ¹¹C-nicotine have demonstrated a decrease in high affinity nicotinic binding sites in post-mortem studies using brain tissues from Alzheimer, Parkinson, and schizophrenic patients. See, Norberg et al., Neurosci. Lett. 72:115-119 (1986); Kellar et al., Brain Res. 436:62-68 (1987); Araujo et al., Neurochem. 50: 1914-1923 (1988); Whitehouse et al., Arch. Neurol. 45: 722-724 (1988); Whitehouse et al., Neurol. 38: 720-723 (1988); London et al., Neurochem. Res. 14: 745-750 (1989); and Freedman et al., Biological Psychiatry, 38: 22-33 (1995). However, there are recognized limitations of using ¹¹C-nicotine as a ligand for measurement of neuronal nicotinic cholinergic receptors in viva. For example, radiotracer uptake is mostly influenced by regional cerebral blood flow (rCBF), and limitations relating to saturability and short ligand-receptor interaction (a reflection of the binding affinity) have been proposed as the major shortcomings of this ligand. See, Villemagne et al., In: Alzheimer's Disease: From Molecular Biology to Therapy, Becker et al. (Eds.), 235-250 (1997). The use of dual tracer using ¹⁵O followed by ¹¹C-nicotine has been proposed as a method to circumvent the cerebral blood flow variations. However, the high non-specific binding has further dampened the effort to use a nicotine-based compound as a viable probe.

Some attempts have been made to use a compound known as ¹¹C ABT-418 as a probe for neuronal nicotinic cholinergic receptors in primates but the results have been disappointing. See. Valette et al., Nucl. Med. Commun. 18:164-168 (1997). The ¹⁸F-labeled analog of a compound known as A-85380 has been investigated for its feasibility as a probe for human neuronal nicotinic cholinergic receptors. See, Valette et al., J. Nucl. Med. 40(8): 1374-1380 (1999). The evaluation of the ¹²³I analog of the compound known as A-85380 as a probe using SPECT has been reported. See, Vaupel et al., Neuroreport 13: 2311-2317 (1998) and Musachio et al., Nucl. Med. Biol. 26: 201-207 (1999). Furthermore, radiolabeled epibatidine, a nicotine analog, has been reported as having potential use to image nicotinic cholinergic receptors. See, U.S. Pat. No. 5,969,144 to London et al. and Villemagne et al., In: Alzheimer's Disease: From Molecular Biology to Therapy, Becker et al. (Eds.), 235-250 (1997). Moreover, ⁷⁶Br labeled compounds have been proposed as useful diagnostic probes. See, also, Muziere et al., Life Sci., 35:1349-1356 (1984); Loc'h et al., Nucl. Med. Bio., 21: 49-55 (1994); Kassiov, J. Lab. Cmp. Radiopharm., 36(3): 259-266 (1995) and Loc'h et al., Nucl. Med. Bio., 23: 813-819 (1996).

It is desirable to provide compounds that act selectively and hence act as probes for the diagnosis of diseases and disorders.

### SUMMARY OF THE INVENTION

The present invention discloses compounds of Formula I: Azabicyclo-N(R₁)—C(═O)—W. Formula I is more fully described in the detailed description. Compounds of Formula I are isotopically labeled compounds and are particularly useful in SPECT (single photon emission computed tomography) and in PET (positron emission tomography).

Embodiments of the invention may include one or more or combination of the following. One embodiment of the present invention provides a compound possessing radiotracer functionalities. Radiolabeled compounds possessing radiotracer functionalities are compounds that possess at least one radioactive isotope as a moiety thereof.

The compound and the method or use of a compound of Formula I, where R₁ is H and Azabicyclo is any one or more of I, II, III, or IV. The compound and the method or use of a compound of Formula I, where R₂ is H or CH₃, each R₃ is H, and R₄ is H.

The compound and the method or use of a compound of Formula I, where W is any one or more of (A), (B), (C), (D), (E), (F), (G), or (H). The compound or the method or use of a compound of Formula I, where W is any one or more of (A), (B), (C), (D), (E), (F), (G), or (H), wherein the variables within each has any definition allowed. For example, and not by way of limitation, W includes any one or more of the following: 4-chlorobenz-1-yl; dibenzo[b,d]thiophene-2-yl; isoquinoline-3-yl; furo[2,3-c]pyridine-5-yl; 1,3-benzodioxole-5-yl; 2,3-dihydro-1,4-benzodioxine-6-yl; 1,3-benzoxazole-5-yl; thieno[2,3-c]pyridine-5-yl; thieno[3,2-c]pyridine-6-yl; [1]benzothieno[3,2-c]pyridine-3-yl; 1,3-benzothiazole-6-yl; thieno[3,4-c]pyridine-6-yl; 2,3-dihydro-1-benzofuran-5-yl; 1-benzofuran-5-yl; furo[3,2-c]pyridine-6-yl; [1]benzothieno[2,3-c]pyridine-3-yl; dibenzo[b,d]furan-2-yl; 1-benzofuran-6-yl; 2-naphthyl; 1H-indole-6-yl; pyrrolo[1,2-c]pyrimidine-3-yl; 1-benzothiophene-5-yl; 1-benzothiophene-5-yl; 1-benzothiophene-6-yl; pyrrolo[1,2-a]pyrazine-3-yl; 1H-indole-6-yl; pyrazino[1,2-a]indole-3-yl; 1,3-benzothiazole-6-yl; [1]benzofuro[2,3-c]pyridine-3-yl; [1]benzofuro[2,3-c]pyridine-3-yl; 2H-chromene-6-yl; indolizine-6-yl; and [1,3]dioxolo[4,5-c]pyridine-6-yl; any of which is optionally substituted as allowed in formula I. For further example, and not by way of limitation, W also includes any one or more of the following: thiophenyl, furanyl, pyrrolyl, oxazolyl, thiazolyl, 1H-pyrazole-yl, isoxazolyl, and isothiazolyl; any of which is optionally substituted as allowed in formula I. More specifically, W includes any one or more of the following: thiophene-2-yl, furan-2-yl, pyrrole-2-yl, 1,3-oxazole-2-yl, 1,3-thiazole-2-yl, isoxazole-3-yl, isothiazole-3-yl; any of which is optionally substituted at the 5 position on the ring as allowed in formula I, and 1,3-oxazole-4-yl, 1,3-oxazole-5-yl, 1,3-thiazole-4-yl, 1,3-thiazole-5-yl; any of which is optionally substituted at the 2 position on the ring as allowed in formula I. One of ordinary skill in the art will recognize how the variables are defined by comparing the named radicals with the different values for W.

The method or use of a compound of Formula I, where the variables of formula I have any definition discussed herein.

The present invention also includes pharmaceutical compositions containing the labeled compounds, and methods to diagnose the identified diseases.

The present invention relates to diagnostic compositions. The present invention relates to compounds that are useful as probes for determining the relative number and/or function of the alpha 7 nAChR. The present invention further includes a method for diagnosing diseases or conditions as discussed herein in a mammal, including human. The method comprises administering to the mammal a detectably labeled compound of Formula I and detecting the binding of that compound to the alpha 7 nAChR.

In another aspect, the present invention relates to a method for administering selective nicotinic receptor subtypes (e.g., alpha 7 nAChR) to a subject, including a human. The method comprises administering a detectably labeled compound of Formula I to the mammal such that the amount administered is detectable but does not reach therapeutic levels and detecting the binding of the compound to the alpha 7 nAChR.

In accordance with the present invention, the compounds that are administered are detected using methods such as PET and SPECT. The present invention allows one skilled in the art of the use of diagnosis tools, such as PET and SPECT, to diagnose a wide variety of conditions and disorders, including conditions and disorders associated with dysfuntion of the central and autonomic nervous system. The present invention is useful in the diagnosis of a wide variety of diseases and disorders where the alpha 7 nAChR is implicated, including any one or more or combination of the following: cognitive and attention deficit symptoms of Alzheimer's, neurodegeneration associated with diseases such as Alzheimer's disease, pre-senile dementia (mild cognitive impairment), senile dementia, schizophrenia, psychosis, attention deficit disorder, attention deficit hyperactivity disorder, depression, anxiety, general anxiety disorder, post traumatic stress disorder, mood and affective disorders, amyotrophic lateral sclerosis, borderline personality disorder, traumatic brain injury, behavioral and cognitive problems in general and associated with brain tumors, AIDS dementia complex, dementia associated with Down's syndrome, dementia associated with Lewy Bodies, Huntington's disease, Parkinson's disease, tardive dyskinesia, Pick's disease, dysregulation of food intake including bulemia and anorexia nervosa, withdrawal symptoms associated with smoking cessation and dependant drug cessation, Gilles de la Tourette's Syndrome, age-related macular degeneration, glaucoma, neurodegeneration associated with glaucoma, diabetic retinopathy, or symptoms associated with pain.

The compounds of Formula I where Azabicyclo is I have asymmetric centers on the quinuclidine ring. The compounds of the present invention include quinuclidines having 3R configuration, 2S, 3R configuration, or 3S configuration and also include racemic mixtures and compositions of varying degrees of streochemical purities. For example, and not by limitation, embodiments of the present invention include compounds of Formula I having the following stereospecificity and substitution:

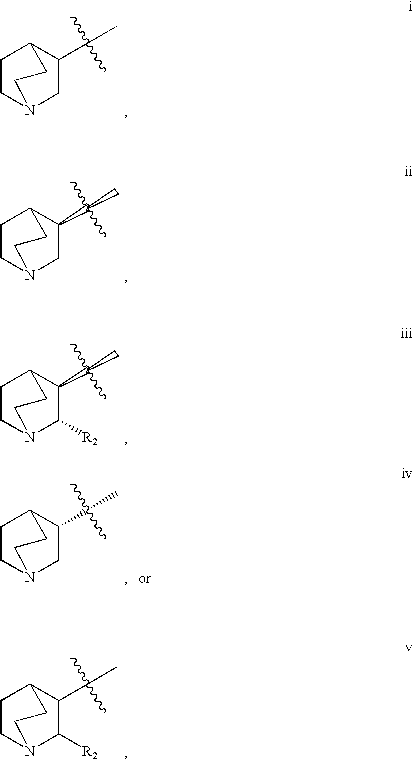

wherein the Azabicyclo (i) is a racemic mixture;

(ii) has the stereochemistry of 3R at C3;

(iii) has the 3R,2S stereochemistry at C3 and C2, respectively, and R₂ is alkyl;

(iv) has the stereochemistry of 3S at C3; or

(v) is a racemic mixture; and for (iii) and (v), R₂ is alkyl.

The compounds of Formula I where Azabicyclo is IV have asymmetric centers on the 7-azabicyclo[2.2.1]heptane ring which can exhibit a number of stereochemical configurations.

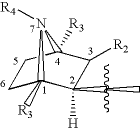

The terms exo and endo are stereochemical prefixes that describe the relative configuration of a substituent on a bridge (not a bridgehead) of a bicyclic system. If a substituent is oriented toward the larger of the other bridges, it is endo. If a substituent is oriented toward the smaller bridge it is exo. Depending on the substitution on the carbon atoms, the endo and exo orientations can give rise to different stereoisomers. For instance, when carbons 1 and 4 are substituted with hydrogen and carbon 2 is bonded to a nitrogen-containing species, the endo orientation gives rise to the possibility of a pair of enantiomers: either the 1S, 2S, 4R isomer or its enantiomer, the 1R, 2R, 4S isomer. Likewise, the exo orientation gives rise to the possibility of another pair of stereoisomers which are diastereomeric and C-2 epimeric with respect to the endo isomers: either the 1R, 2S, 4S isomer or its enantiomer, the 1S, 2R, 4R isomer. The compounds of this invention exist in the exo orientation. For example, when R₂═R₃═H, the absolute stereochemistry is exo-(1S, 2R, 4R).

The compounds of the present invention have the exo orientation at the C-2 carbon and S configuration at the C-1 carbon and the R configuration at the C-2 and the C-4 carbons of the 7-azabicyclo[2.2.1]heptane ring. Unexpectedly, the inventive compounds exhibit much higher activity relative to compounds lacking the exo 2R, stereochemistry. For example, the ratio of activities for compounds having the exo 2R configuration to other stereochemical configurations may be greater than about 100:1. Although it is desirable that the stereochemical purity be as high as possible, absolute purity is not required. For example, pharmaceutical compositions can include one or more compounds, each having an exo 2R configuration, or mixtures of compounds having exo 2R and other configurations. In mixtures of compounds, those species possessing stereochemical configurations other than exo 2R act as diluents and tend to lower the activity of the pharmaceutical composition. Typically, pharmaceutical compositions including mixtures of compounds possess a larger percentage of species having the exo 2R configuration relative to other configurations.

The compounds of Formula I where Azabicyclo is II have asymmetric center(s) on the [2.2.1] azabicyclic ring at C3 and C4. The scope of this invention includes the separate stereoisomers of Formula I being endo-4S, endo-4R, exo-4S, exo-4R:

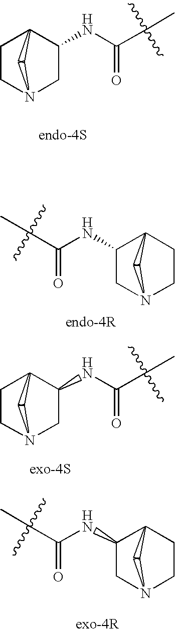

The endo isomer is the isomer where the non-hydrogen substituent at C3 of the [2.2.1] azabicyclic compound is projected toward the larger of the two remaining bridges. The exo isomer is the isomer where the non-hydrogen substituent at C3 of the [2.2.1] azabicyclic compound is projected toward the smaller of the two remaining bridges. Thus, there can be four separate isomers: exo-4(R), exo-4(S), endo-4(R), and endo-4(S). Some embodiments of compounds of Formula I for when Azabicyclo is HI include racemic mixtures where R₂ is H or is at C2 or C6 and is alkyl; or Azabicyclo II has the exo-4(S) stereochemistry and R₂ has any definition discussed herein and is bonded at any carbon discussed herein.

The compounds of Formula I where Azabicyclo is III have asymmetric center(s) on the [3.2.1] azabicyclic ring at C3 and C5. The scope of this invention includes the separate stereoisomers of Formula I being endo-3S, 5R, endo-3R, 5S, exo-3R, 5R, exo-3S, 5S:

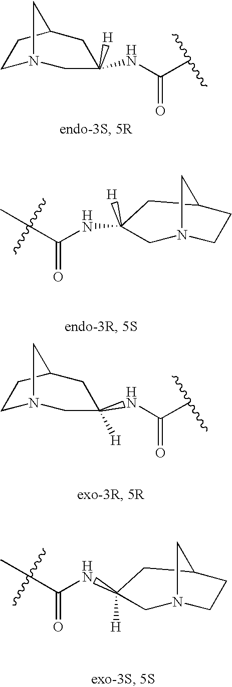

Another group of compounds of Formula I (Azabicyclo III) includes compounds where Azabicyclo III moiety has the stereochemistry of 3R, 5R, or is a racemic mixture and R₂ is either H or alkyl at either C2 and/or C4.

Stereoselective syntheses and/or subjecting the reaction product to appropriate purification steps produce substantially enantiomerically pure materials. Suitable stereoselective synthetic procedures for producing enantiomerically pure materials are well known in the art, as are procedures for purifying racemic mixtures into enantiomerically pure fractions.

The compounds of the present invention having the specified stereochemistry above have different levels of activity and that for a given set of values for the variable substitutuents one isomer may be preferred over the other isomers. Although it is desirable that the stereochemical purity be as high as possible, absolute purity is not required. It is preferred to carry out stereoselective syntheses and/or to subject the reaction product to appropriate purification steps so as to produce substantially enantiomerically pure materials. Suitable stereoselective synthetic procedures for producing enantiomerically pure materials are well known in the art, as are procedures for purifying racemic mixtures into enantiomerically pure fractions.

The invention includes isotopically-labeled compounds, wherein at least one atom of formula I is an atom having an atomic mass or mass number different from the atomic mass or mass number most abundantly found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹³N, ¹⁵O, ¹⁸F, ^{99m}Tc, ¹²³I, and ¹²⁵I. Compounds of the present invention and pharmaceutically acceptable salts and prodrugs of said compounds that contain the aforementioned isotopes and/or other isotopes of other atoms are within the scope of the invention. Isotopically-labeled compounds of the present invention are useful in drug and/or substrate tissue distribution and target occupancy assays. For example, isotopically labeled compounds are particularly useful in SPECT (single photon emission computed tomography) and in PET (positron emission tomography).

SPECT acquires information on the concentration of isotopically labeled compounds introduced to a mammal's body. SPECT dates from the early 1960's, when the idea of emission traverse section tomography was introduced by D. E. Kuhl and R. Q. Edwards prior to either PET, x-ray CT, or MRI. In general, SPECT requires isotopes that decay by electron capture and/or gamma emission. Examples of viable SPECT isotopes include, but are not limited to, 123-iodine (¹²³I) and 99m-technetium (^{99m}Tc). A mammal is injected with a radioactively labeled agent at tracer doses. Tracer doses are doses sufficient to allow the diagnosis to occur (e.g., to allow detection of the labeled compound) but are not sufficient to have a therapeutic effect on the mammal. The nuclear decay resulting in the emission of a single gamma ray which passes through the tissue and is measured externally with a SPECT camera. The uptake of radioactivity reconstructed by computers as a tomogram shows tissue distribution in cross-sectional images.

PET is a technique for measuring the concentrations of positron-emitting isotopes within the tissues. Like SPECT, these measurements are, typically, made using PET cameras outside of the living mammals. PET can be broken down into several steps including, but not limited to, synthesizing a compound to include a positron-emitting isotope; administering the isotopically labeled compound to a mammal; and imaging the distribution of the positron activity as a function of time by emission tomography. PET is described, for example, by Alavi et al. in Positron Emission Tomography, published by Alan R. Liss, Inc. in 1985.

Positron-emitting isotopes used in PET include, but are not limited to, Carbon-11, Nitrogen-13, Oxygen-15, and Fluorine-18. In general, positron-emitting isotopes should have short half-lives to help minimize the long-term radiation exposure that a mammal receives from high dosages required during PET imaging.

In certain instances, PET imaging can be used to measure the binding kinetics of compounds of this invention with alpha 7 nAChRs. For example, administering an isotopically labeled compound of the invention that penetrates into the body and binds to an alpha 7 nAChR creates a baseline PET signal which can be monitored while administering a second, different, non-isotopically labeled compound. The baseline PET signal will decrease as the non-isotopically labeled compound competes for the binding to the alpha 7 nAChR.

In general, compounds of the present invention are useful in performing PET or SPECT and are those which penetrate the blood-brain barrier, exhibit high selectivity and selective affinity to alpha 7 nAChRs, and are eventually metabolized. Compounds that are non-selective, exhibit excessive or small affinity for alpha 7 nAChRs, or exhibit low penetration through the blood-brain barrier are, generally, not useful in studying brain receptor binding kinetics with respect to alpha 7 nAChRs. Compounds that are not metabolized may harm the patient. Methods for determining the blood-brain penetration and the affinity for alpha 7 nAChRs are described below.

The compounds of the present invention may be administered by any suitable route, preferably in the form of a pharmaceutical composition adapted to such a route, and in a dose effective to image and desirably quantify the nAChRs in the brain. Preferably, the compounds are administered intravenously to minimize metabolism before the compound enters the brain. The amount of the compounds of the present invention required to image or quantify the α7 nAChRs in the brain will be readily ascertained by one of ordinary skill in the nuclear medicine art taking into account the specific activity of the compound and the radiation dosimetry. As is known by those skilled in the nuclear medicine art, the number of milliCuries of the radiolabeled compounds to be administered for the PET or SPECT scan will be limited by the dosimetry, whereas the mass of compound to be administered (e.g., μg/kg or mg/kg of body weight of the patient) is calculated based on the specific activity of the synthesized compound, i.e., the amount of radioactivity/mass, of radiolabeled compound. It will be appreciated that because of the short half-life of the radioisotopes, e.g., about 2 hours for ¹⁸F and about 20 minutes for ¹¹C, it is often necessary to make the radiolabeled compound at or near the site of administration. For ¹²³I, the half-life is slightly longer, being about 13 hours. The specific activity of the compounds must then be ascertained in order to calculate the proper dosing. Such techniques are well known to those skilled in the art.

By way of illustration, and not in limitation, it has been found that in mice, the microCuries of radioisotopes should be about 200 to 300, and in baboons the milliCuries should be about 5. In keeping with that determination, the injected mass of the radiolabeled agonist should be less than 1 μg/kg. Further, for a radiolabeled agonist of 2000 milliCuries/micromole, about 5 millicuries of radiation should be administered to a 70 kg patient. It is preferable not to use a radiolabeled compound of less than 1500 milliCuries/micromole.

Preferred compounds for isotopic labeling and use in performing PET include any one or more or combination of the following:

N-[(3R)-1-azabicyclo[2.2.2]oct-3-yl]-1-benzofuran-5-[¹¹C]carboxamide;

N-[(2S,3R)-2-methyl-1-azabicyclo[2.2.2]oct-3-yl]-1-benzofuran-5-[¹¹C]carboxamide;

N-[(3R,5R)-1-azabicyclo[3.2.1]oct-3-yl]-1-benzofuran-5-[¹¹C]carboxamide;

N-[(3R)-1-azabicyclo[2.2.2]oct-3-yl]-2-methyl-1-benzofuran-5-[¹¹C]carboxamide;

N-[(2S,3R)-2-methyl-1-azabicyclo[2.2.2]oct-3-yl]-2-methyl-1-benzofuran-5-[¹¹C]carboxamide;

N-[(3R,5R)-1-azabicyclo[3.2.1 loct-3-yl]-2-methyl-1-benzofuran-5-[¹¹C]carboxamide;

N-[(3R)-1-azabicyclo[2.2.2]oct-3-yl]furo[2,3-c]pyridine-5-[¹¹C]carboxamide;

N-[(2S,3R)-2-methyl-1-azabicyclo[2.2.2]oct-3-yl]furo[2,3-c]pyridine-5-[¹¹C]carboxamide;

N-[(2R)-7-azabicyclo[2.2.1]hept-2-yl]furo[2,3-c]pyridine-5-[¹¹C]carboxamide;

N-[(3R,5R)-1-azabicyclo[3.2.1]oct-3-yl]furo[2,3-c]pyridine-5-[¹¹C]carboxamide;

N-[(3S)-1-azabicyclo[2.2.2]oct-3-yl]-3-methylfuro[2,3-c]pyridine-5-[¹¹C]carboxamide;

N-[(3R)-1-azabicyclo[2.2.2]oct-3-yl]-5-bromothiophene-2-[¹¹C]carboxamide;

5-bromo-N-[(2S,3R)-2-methyl-1-azabicyclo[2.2.2]oct-3-yl]thiophene-2-[¹¹C]carboxamide;

N-[(3R)-1-azabicyclo[2.2.2]oct-3-yl]-5-pyridin-2-ylthiophene-2-[¹¹C]carboxamide;

N-[(2S,3R)-2-methyl-1-azabicyclo[2.2.2]oct-3-yl]-5-pyridin-2-ylthiophene-2-[¹¹C]carboxamide;

N-[(3R)-1-azabicyclo[2.2.2]oct-3-yl]-5-(methylthio)thiophene-2-[¹¹C]carboxamide;

N-[(2S,3R)-2-methyl-1-azabicyclo[2.2.2]oct-3-yl]-5-(methylthio)thiophene-2-[¹¹C]carboxamide;

N-[(3R)-1-azabicyclo[2.2.2]oct-3-yl]-5-phenylthiophene-2-[¹¹C]carboxamide;

N-[(3R)-1-azabicyclo[2.2.2]oct-3-yl]-5-methoxythiophene-2-[¹¹C]carboxamide;

N-[(2S,3R)-2-methyl-1-azabicyclo[2.2.2]oct-3-yl]-5-methoxythiophene-2-[¹¹C]carboxamide;

N-[(3R)-1-azabicyclo[2.2.2]oct-3-yl]-5-nitrothiophene-2-[¹¹C]carboxamide;

N-[(2S,3R)-2-methyl-1-azabicyclo[2.2.2]oct-3-yl]-5-nitrothiophene-2-[¹¹C]carboxamide;

N-[(3R)-1-azabicyclo[2.2.2]oct-3-yl]-5-(2-[¹⁸F]fluorophenyl)-2-furamide;

5-(2-[¹⁸F]fluorophenyl)-N-[(2S,3R)-2-methyl-1-azabicyclo[2.2.2]oct-3-yl]-2-furamide; or pharmaceutically acceptable salts thereof.

Preferred compounds for isotopic labeling and use in performing SPECT include any one or more or combination of the following:

N-[(3R)-1-azabicyclo[2.2.2]oct-3-yl]-4-[1231]iodo-1H-pyrazole-1-carboxamide;

N-[(2S,3R)-2-methyl-1-azabicyclo[2.2.2]oct-3-yl]-4-[¹²³I]iodo-1H-pyrazole-1-carboxamide;

N-[(3R,5R)-1-azabicyclo[3.2.1]oct-3-yl]-4-[123]iodo-1H-pyrazole-1-carboxamide; or pharmaceutically acceptable salts thereof.

In other embodiments, nuclear magnetic resonance spectroscopy (NMR or also referenced as MRS) imaging can be used to detect the overall concentration of a compound or fragment thereof containing nuclei with a specific spin. In general, the isotopes useful in MRS imaging include, but are not limited to, hydrogen-1, carbon-13, phosphorus-31, and fluorine-19. Examples of compounds useful for MRS include any one or more or combination of the following:

N-[(3R)-1-azabicyclo[2.2.2]oct-3-yl]-1-benzofuran-5-[¹³C]carboxamide;

N-[(2S,3R)-2-methyl-1-azabicyclo[2.2.2]oct-3-yl]-1-benzofuran-5-[¹³C]carboxamide;

N-[(3R,5R)-1-azabicyclo[3.2.1]oct-3-yl]-1-benzofuran-5-[¹³C]carboxamide;

N-[(3R)-1-azabicyclo[2.2.2]oct-3-yl]-2-methyl-1-benzofuran-5-[¹³C]carboxamide;

N-[(2S,3R)-2-methyl-1-azabicyclo[2.2.2]oct-3-yl]-2-methyl-1-benzofuran-5-[¹³C]carboxamide;

N-[(3R,5R)-1-azabicyclo[3.2. I]oct-3-yl]-2-methyl-1-benzofuran-5-[¹³C]carboxamide;

N-[(3R)-1-azabicyclo[2.2.2]oct-3-yl]furo[2,3-c]pyridine-5-[¹³C]carboxamide;

N-[(2S,3R)-2-methyl-1-azabicyclo[2.2.2]oct-3-yl]furo[2,3-c]pyridine-5-[¹³C]carboxamide;

N-[(2R)-7-azabicyclo[2.2.1]hept-2-yl]furo[2,3-c]pyridine-5-[¹³C]carboxamide;

N-[(3R,5R)-1-azabicyclo[3.2.1]oct-3-yl]furo[2,3-c]pyridine-5-[¹³C]carboxamide;

N-[(3S)-1-azabicyclo[2.2.2]oct-3-yl]-3-methylfuro[2,3-c]pyridine-5-[¹³C]carboxamide;

N-[(3R)-1-azabicyclo[2.2.2]oct-3-yl]-5-bromothiophene-2-[¹³C]carboxamide;

5-bromo-N-[(2S,3R)-2-methyl-1-azabicyclo[2.2.2]oct-3-yl]thiophene-2-[¹³C]carboxamide;

N-[(3R)-1-azabicyclo[2.2.2]oct-3-yl]-5-pyridin-2-ylthiophene-2-[¹³C]carboxamide;

N-[(2S,3R)-2-methyl-1-azabicyclo[2.2.2]oct-3-yl]-5-pyridin-2-ylthiophene-2-[¹³C]carboxamide;

N-[(3R)-1-azabicyclo[2.2.2]oct-3-yl]-5-(methylthio)thiophene-2-[¹³C]carboxamide;

N-[(2S,3R)-2-methyl-1-azabicyclo[2.2.2]oct-3-yl]-5-(methylthio)thiophene-2-[¹³C]carboxamide;

N-[(3R)-1-azabicyclo[2.2.2]oct-3-yl]-5-phenylthiophene-2-[¹³C]carboxamide;

N-[(3R)-1-azabicyclo[2.2.2]oct-3-yl]-5-methoxythiophene-2-[¹³C]carboxamide;

N-[(2S,3R)-2-methyl-1-azabicyclo[2.2.2]oct-3-yl]-5-methoxythiophene-2-[¹³C]carboxamide;

N-[(3R)-1-azabicyclo[2.2.2]oct-3-yl]-5-nitrothiophene-2-[¹³C]carboxamide;

N-[(2S,3R)-2-methyl-1-azabicyclo[2.2.2]oct-3-yl]-5-nitrothiophene-2-[¹³C]carboxamide;

N-[(3R)-1-azabicyclo[2.2.2]oct-3-yl]-5-(2-[⁹F]fluorophenyl)-2-furamide;

5-(2-[¹⁹F]fluorophenyl)-N-[(2S,3R)-2-methyl-1-azabicyclo[2.2.2]oct-3-yl]-2-furamide;

Further, substitution with heavier isotopes such as deuterium, i.e., ²H, can afford certain therapeutic advantages resulting from greater metabolic stability, for example, increased in vivo half-life or reduced dosage requirements and, hence, may be preferred in some circumstances.

Isotopically labeled compounds of Formula I can generally be prepared by carrying out the synthetic procedures described herein by substituting an isotopically labeled reagent for a non-isotopically labeled reagent. Isotopically labeled reagents are described, for example, by Langstrom in Acta Chem. Scand. S37: 147 (1990). Introducing ¹¹C-labeled agonists of nAChR has been described in Dolle, Frederic, et al, J. Labelled Cps Radiopharm., 2001; 44: 785-795. For a general discussion of nuclear imaging, see, “Nuclear Imaging in Drug Discovery, Development, and Approval,” H. D. Burns, et al. (Eds).

Further aspects and embodiments of the invention may become apparent to those skilled in the art from a review of the following detailed description, taken in conjunction with the examples and the appended claims. While the invention is susceptible of embodiments in various forms, described hereafter are specific embodiments of the invention with the understanding that the present disclosure is intended as illustrative, and is not intended to limit the invention to the specific embodiments described herein.

### DETAILED DESCRIPTION OF THE INVENTION

Surprisingly, we have found that α7 nAChR agonists can be used as diagnostic tools as discussed herein. Alpha 7 nAChR agonists within the scope of the present invention include compounds of Formula I:

Azabicyclo-N(R₁)—C(═O)—W Formula I

wherein Azabicyclo is

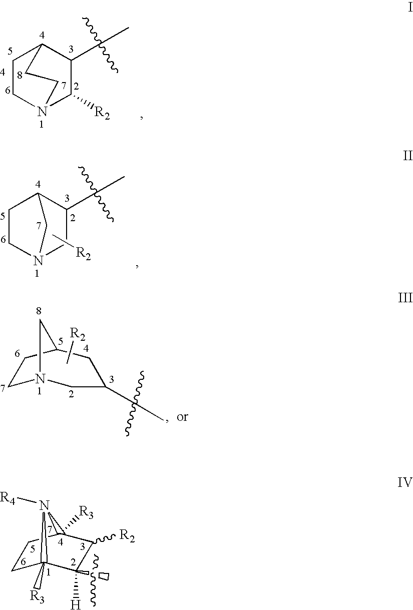

R₁ is H;

R₂ is H or alkyl;

Each R₃ is independently H, alkyl, or substituted alkyl;

R₄ is H, alkyl, an amino protecting group, or an alkyl group having 1-3 substituents selected from F, Cl, Br, 1, —OH, —CN, —NH₂, —NH(alkyl), or —N(alkyl)₂;

Lower alkyl is both straight- and branched-chain moieties having from 1-4 carbon atoms, unless otherwise specified;

Lower haloalkyl is lower alkyl having 1 to (2n+1) substituent(s) independently selected from F, Cl, Br, or I where n is the maximum number of carbon atoms in the moiety;

Lower substituted alkyl is lower alkyl having 0-3 substituents independently selected from F, Cl, Br, or I and further having 1 substituent selected from R₅, R₆, —CN, —NO₂, —OR₈, —SR₈, —N(R₈)₂, —C(O)R₈, —C(O)OR₈, —C(S)R₈, —C(O)N(R₈)₂, —NR₈C(O)N(R₈)₂, —NR₈C(O)R₈, —S(O)R₈, —S(O)₂R₈, —OS(O)₂R₈, —S(O)₂N(R₈)₂, —NR₈S(O)₂R₈, phenyl, or phenyl having 1 substituent selected from R₉ and further having 0-3 substituents independently selected from F, Cl, Br, or I;

Alkyl is both straight- and branched-chain moieties having from 1-6 carbon atoms;

Haloalkyl is alkyl having 1 to (2n+1) substituent(s) independently selected from F, Cl, Br, or I where n is the maximum number of carbon atoms in the moiety;

Substituted alkyl is alkyl having 0-3 substituents independently selected from F, Cl, Br, or I and further having 1 substituent selected from R₅, R₆, —CN, —NO₂, —OR₈, —SR₈, —N(R₈)₂, —C(O)R₈, —C(O)OR₈, —C(S)R₈, —C(O)N(R₈)₂, —NR₈C(O)N(R₈)₂, —NR₈C(O)R₈, —S(O)R₈, —S(O)₂R₈, —OS(O)₂R₈, —S(O)₂N(R₈)₂, —NR₈S(O)₂R₈, phenyl, or phenyl having 1 substituent selected from R₉ and further having 0-3 substituents independently selected from F, Cl, Br, or I;

Alkenyl is straight- and branched-chain moieties having from 2-6 carbon atoms and having at least one carbon-carbon double bond;

Haloalkenyl is alkenyl having 1 to (2n-1) substituent(s) independently selected from F, Cl, Br, or I where n is the maximum number of carbon atoms in the moiety;

Substituted alkenyl is alkenyl having 0-3 substituents independently selected from F, or Cl, and further having 1 substituent selected from R₅, R₆, —CN, —NO₂, —OR₈, —SR₈, —N(R₈)₂, —C(O)R₈, —C(O)OR₈, —C(S)R₈, —C(O)N(R₈)₂, —NR₈C(O)N(R₈)₂, —NR₈C(O)R₈, —S(O)R₈, —S(O)₂R₈, —OS(O)₂R₈, —S(O)₂N(R₈)₂, —NR₈S(O)₂R₈, phenyl, or phenyl having 1 substituent selected from R₉ and further having 0-3 substituents independently selected from F, Cl, Br, or I;

Alkynyl is straight- and branched-chained moieties having from 2-6 carbon atoms and having at least one carbon-carbon triple bond;

Haloalkynyl is alkynyl having 1 to (2n-3) substituent(s) independently selected from F, Cl, Br, or I where n is the maximum number of carbon atoms in the moiety;

Substituted alkynyl is alkynyl having 0-3 substituents independently selected from F, or Cl, and further having 1 substituent selected from R₅, R₆, —CN, —NO₂, —OR₈, —SR₈, —N(R₈)₂, —C(O)R₈, —C(O)OR₈, —C(S)R₈, —C(O)N(R₈)₂, —NR₈C(O)N(R₈)₂, —NR₈C(O)R₈, —S(O)R₈, —S(O)₂R₈, —OS(O)₂R₈, —S(O)₂N(R₈)₂, —NR₈S(O)₂R₈, phenyl, or phenyl having 1 substituent selected from R₉ and further having 0-3 substituents independently selected from F, Cl, Br, or I;

Cycloalkyl is a cyclic alkyl moiety having from 3-6 carbon atoms;

Halocycloalkyl is cycloalkyl having 1-4 substituents independently selected from F, or Cl;

Substituted cycloalkyl is cycloalkyl having 0-3 substituents independently selected from F, or Cl, and further having 1 substituent selected from R₅, R₆, —CN, —NO₂, —OR₈, —SR₈, —N(R₈)₂, —C(O)R₈, —C(O)OR₈, —C(S)R₈, —C(O)N(R₈)₂, —NR₈C(O)N(R₈)₂, —NR₈C(O)R₈, —S(O)R₈, —S(O)₂R₈, —OS(O)₂R₈, —S(O)₂N(R₈)₂, —NR₈S(O)₂R₈, phenyl, or phenyl having 1 substituent selected from R₉ and further having 0-3 substituents independently selected from F, Cl, Br, or I;

Heterocycloalkyl is a cyclic moiety having 4-7 atoms with 1-2 atoms within the ring being —S—, —N(R₁₀)-, or —O—;

Haloheterocycloalkyl is heterocycloalkyla having 1-4 substituents independently selected from F, or Cl;

Substituted heterocycloalkyl is heterocycloalkyl having 0-3 substituents independently selected from F, or Cl, and further having 1 substituent selected from R₅, R₆, —CN, —NO₂, —OR₈, —SR₈, —N(R₈)₂, —C(O)R₈, —C(O)OR₈, —C(S)R₈, —C(O)N(R₈)₂, —NR₈C(O)N(R₈)₂, —NR₈C(O)R₈, —S(O)R₈, —S(O)₂R₈, —OS(O)₂R₈, —S(O)₂N(R₈)₂, —NR₈S(O)₂R₈, phenyl, or phenyl having 1 substituent selected from R₉ and further having 0-3 substituents independently selected from F, Cl, Br, or I;

Lactam heterocycloalkyl is a cyclic moiety having from 4-7 atoms with one atom being only nitrogen with the bond to the lactam heterocycloalkyl thru said atom being only nitrogen and having a ═O on a carbon adjacent to said nitrogen, and having up to 1 additional ring atom being oxygen, sulfur, or nitrogen and further having 0-2 substituents selected from F, Cl, Br, I, or R₇ where valency allows;

Aryl is phenyl, substituted phenyl, naphthyl, or substituted naphthyl;

Substituted phenyl is a phenyl either having 1-4 substituents independently selected from F, Cl, Br, or I, or having 1 substituent selected from R₁₁ and 0-3 substituents independently selected from F, Cl, Br, or I;

Substituted naphthyl is a naphthalene moiety either having 1-4 substituents independently selected from F, Cl, Br, or I, or having 1 substituent selected from R₁₁ and 0-3 substituents independently selected from F, Cl, Br, or I, where the substitution can be independently on either only one ring or both rings of said naphthalene moiety;

Substituted phenoxy is a phenoxy either having 1-3 substituents independently selected from F, Cl, Br, or I, or having 1 substituent selected from R₁₁ and 0-2 substituents independently selected from F, Cl, Br, or I;

R₅ is 5-membered heteroaromatic mono-cyclic moieties containing within the ring 1-3 heteroatoms independently selected from the group consisting of —O—, ═N—, —N(R₁₀)—, and —S—, and having 0-1 substituent selected from R₉ and further having 0-3 substituents independently selected from F, Cl, Br, or I, or R₅ is 9-membered fused-ring moieties having a 6-membered ring fused to a 5-membered ring and having the formula

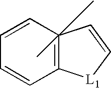

wherein L₁ is O, S, or NR₁₀,

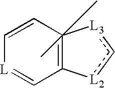

wherein L is CR₁₂ or N, L₂ and L₃ are independently selected from CR₁₂, C(R₁₂)₂, O, S, N, or NR₁₀, provided that both L₂ and L₃ are not simultaneously 0, simultaneously S, or simultaneously O and S, or

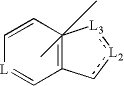

wherein L is CR₁₂ or N, and L₂ and L₃ are independently selected from CR₁₂, O, S, N, or NR₁₀, and each 9-membered fused-ring moiety having 0-1 substituent selected from R₉ and further having 0-3 substituent(s) independently selected from F, Cl, Br, or I, wherein the R₅ moiety attaches to other substituents as defined in formula I at any position as valency allows;

R₆ is 6-membered heteroaromatic mono-cyclic moieties containing within the ring 1-3 heteroatoms selected from ═N— and having 0-1 substituent selected from R₉ and 0-3 substituent(s) independently selected from F, Cl, Br, or I, or R₆ is 10-membered heteroaromatic bi-cyclic moieties containing within one or both rings 1-3 heteroatoms selected from ═N—, including, but not limited to, quinolinyl or isoquinolinyl, each 10-membered fused-ring moiety having 0-1 substituent selected from R₉ and 0-3 substituent(s) independently selected from F, Cl, Br, or I, wherein the R₆ moiety attaches to other substituents as defined in formula I at any position as valency allows;

R₇ is alkyl, substituted alkyl, haloalky, —OR₁₁, —CN, —NO₂, —N(R₈)₂;

Each R₈ is independently H, alkyl, cycloalkyl, heterocycloalkyl, alkyl substituted with 1 substituent selected from R₁₃, cycloalkyl substituted with I substituent selected from R₁₃, heterocycloalkyl substituted with 1 substituent selected from R₁₃, haloalkyl, halocycloalkyl, haloheterocycloalkyl, phenyl, or substituted phenyl;

R₉ is alkyl, cycloalkyl, heterocycloalkyl, haloalkyl, halocycloalkyl, haloheterocycloalkyl, —OR₁₄, —SR₁₄, —N(R₁₄)₂, —C(O)R₁₄, —C(O)N(R₁₄)₂, —CN, —NR₁₄C(O)R₁₄, —S(O)₂N(R₁₄)₂, —NR₁₄S(O)₂R₁₄, —NO₂, alkyl substituted with 1-4 substituent(s) independently selected from F, Cl, Br, I, or R₁₃, cycloalkyl substituted with 1-4 substituent(s) independently selected from F, Cl, Br, I, or R₁₃, or heterocycloalkyl substituted with 1-4 substituent(s) independently selected from F, Cl, Br, I, or R₁₃;

R₁₀ is H, alkyl, haloalkyl, substituted alkyl, cycloalkyl, halocycloalkyl, substituted cycloalkyl, phenyl, or phenyl having 1 substituent selected from R₇ and further having 0-3 substituents independently selected from F, Cl, Br, or I;

Each R₁ is independently H, alkyl, cycloalkyl, heterocycloalkyl, haloalkyl, halocycloalkyl, or haloheterocycloalkyl;

Each R₁₂ is independently H, F, Cl, Br, I, alkyl, cycloalkyl, heterocycloalkyl, haloalkyl, halocycloalkyl, haloheterocycloalkyl, substituted alkyl, substituted cycloalkyl, substituted heterocycloalkyl, —CN, —NO₂, —OR₁₄, —SR₁₄, —N(R₁₄)₂, —C(O)R₁₄, —C(O)N(R₁₄)₂, —NR₁₄C(O)R₁₄, —S(O)₂N(R₁₄)₂, —NR₁₄S(O)₂RR₁₄, or a bond directly or indirectly attached to the core molecule, provided that there is only one said bond to the core molecule within the 9-membered fused-ring moiety, further provided that where valency allows the fused-ring moiety has 0-1 substituent selected from alkyl, cycloalkyl, heterocycloalkyl, haloalkyl, halocycloalkyl, haloheterocycloalkyl, substituted alkyl, substituted cycloalkyl, substituted heterocycloalkyl, —OR₁₄, —SR₁₄, —N(R₁₄)₂, —C(O)R₁₄, —NO₂, —C(O)N(R₁₄)₂, —CN, —NR₁₄C(O)R₁₄, —S(O)₂N(R₁₄)₂, or —NR₁₄S(O)₂R₁₄, and further provided that the fused-ring moiety has 0-3 substituent(s) selected from F, Cl, Br, or I;

R₁₃ is —OR₁₄, —SR₁₄, —N(R₁₄)₂, —C(O)R₁₄, —C(O)N(R₁₄)₂, —CN, —CF₃, —NR₁₄C(O)R₁₄, —S(O)₂N(R₁₄)₂, —NR₁₄S(O)₂R₁₄, or —NO₂;

Each R₁₄ is independently H, alkyl, cycloalkyl, heterocycloalkyl, haloalkyl, halocycloalkyl, or haloheterocycloalkyl;

wherein W is (A):

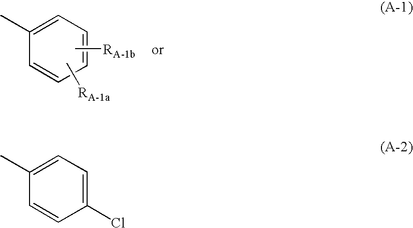

R_{A-1a} is H, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, aryl, —R₅, R₆, —OR_{A-3}, —OR_{A-4}, —SR_{A-3}, F, Cl, Br, I, —N(R_{A-3})₂, —N(R_{A-5})₂, —C(O)R_{A-3}, —C(O)R_{A-5}, —CN, —C(O)N(R_{A-3})₂, —C(O)N(R_{A-6})₂, —NR_{A-3}C(O)R_{A-3}, —S(O)R_{A-3}, —OS(O)₂R_{A-3}, —NR_{A-3}S(O)₂R_{A-3}, —NO₂, and —N(H)C(O)N(H)R_{A-3};

R_{A-1b} is —O—R_{A-3}, —S-R_{A-3}, —S(O)—R_{A-3}, —C(O)—R_{A-7}, and alkyl substituted on the ω carbon with R_{A-7} where said co carbon is determined by counting the longest carbon chain of the alkyl moiety with the C-1 carbon being the carbon attached to the phenyl ring attached to the core molecule and the ω carbon being the carbon furthest from said C-1 carbon;

Each R_{A-3} is independently selected from H, alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;

R_{A-4} is selected from cycloalkyl, halocycloalkyl, substituted cycloalkyl, heterocycloalkyl, haloheterocycloalkyl, or substituted heterocycloalkyl;

Each R_{A-5} is independently selected from cycloalkyl, halocycloalkyl, substituted cycloalkyl, heterocycloalkyl, haloheterocycloalkyl, substituted heterocycloalkyl, R₅, R₆, phenyl, or substituted phenyl;

Each R_{A-6} is independently selected from alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;

R_{A-7} is selected from aryl, R₅, or R₆;

wherein W is (B):

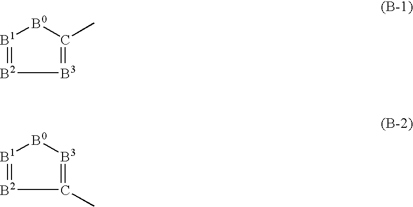

B⁰ is —O—, —S—, or —N(R_{B-0})—;

B¹ and B² are independently selected from ═N—, or ═C(R_{B-1})—;

B³ is ═N—, or ═CH—, provided that when both B¹ and B² are ═C(R_{B-1})— and B³ is ═CH—, only one ═C(R_{B-1})— can be ═CH—, and further provided that when B⁰ is —O—, B² is ═C(R_{B-1})— and B³ is ═C(H)—, B¹ cannot be ═N—,

R_{B-0} is H, alkyl, cycloalkyl, heterocycloalkyl, haloalkyl, halocycloalkyl, haloheterocycloalkyl, substituted alkyl, limited substituted alkyl, substituted cycloalkyl, substituted heterocycloalkyl, or aryl, and provided that when B is (B-2) and B³ is ═N— and B⁰ is N(R_{B-0}), R_{B-0} cannot be phenyl or substituted phenyl;

R_{B-1} is H, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, limited substituted alkyl, limited substituted alkenyl, limited substituted alkynyl, aryl, —OR_{B-2}, —OR_{B-3}, —SR_{B-2}, —SR_{B-3}, F, Cl, Br, I, —N(R_{B-2})₂, —N(R_{B-3})₂, —C(O)R_{B-2}, —C(O)R_{B-3}, —C(O)N(R_{B-2})₂, —C(O)N(R_{B-3})₂, —CN, —NR_{B-2}C(O)R_{B-4}, —S(O)₂N(R_{B-2})₂, —OS(O)₂R_{B-4}, —S(O)₂R_{B-2}, —S(O)₂R_{B-3}, —NR_{B-2}S(O)₂R_{B-2}, —N(H)C(O)N(H)R_{B-2}, —NO₂, R₅, and R₆;

Each R_{B-2} is independently H, alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;

Each R_{B-3} is independently H, alkyl, haloalkyl, limited substituted alkyl, cycloalkyl, halocycloalkyl, substituted cycloalkyl, heterocycloalkyl, haloheterocycloalkyl, substituted heterocycloalkyl;

R_{B-4} is independently H, alkyl, cycloalkyl, heterocycloalkyl, haloalkyl, halocycloalkyl, or haloheterocycloalkyl;

wherein W is (C):

(C) is a six-membered heterocyclic ring system having 1-2 nitrogen atoms or a 10-membered bicyclic-six-six-fused-ring system having up to two nitrogen atoms within either or both rings, provided that no nitrogen is at a bridge of the bicyclic-six-six-fused-ring system, and further having 1-2 substitutents independently selected from R_{C-1};

Each R_{C-1} is independently H, F, Cl, Br, I, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocyloalkyl, substituted heterocycloalkyl, lactam heterocycloalkyl, phenyl, substituted phenyl, —NO₂, —CN, —OR_{C-2}, —SR_{C-2}, —SOR_{C-2}, —SO₂R_{C-2}, —NR_{C-2}C(O)R_{C-3}, —NR_{C-2}C(O)R_{C-2}, —NR_{C-2}C(O)Rc₄, —N(R_{C-2})₂, —C(O)R_{C-2}, —C(O)₂R_{C-2}, —C(O)N(R_{C-2})₂, —SCN, —NR_{C-2}C(O)R_{C-2}, —S(O)N(R_{C-2})₂, —S(O)₂N(R_{C-2})₂, —NR_{C-2}S(O)₂R_{C-2}, R₅, or R₆;

Each R_{C-2} is independently H, alkyl, cycloalkyl, heterocycloalkyl, alkyl substituted with 1 substituent selected from R_{C-5}, cycloalkyl substituted with 1 substituent selected from R_{C-5}, heterocycloalkyl substituted with 1 substituent selected from R_{C-5}, haloalkyl, halocycloalkyl, haloheterocycloalkyl, phenyl, or substituted phenyl;

Each R_{C-3} is independently H, alkyl, or substituted alkyl;

R_{C-4} is H, alkyl, an amino protecting group, or an alkyl group having 1-3 substituents selected from F, Cl, Br, 1, —OH, —CN, —NH₂, —NH(alkyl), or —N(alkyl)₂;

R_{C-5} is —CN, —CF₃, —NO₂, —OR_{C-6}, —SR_{C-6}, —N(R_{C-6})₂, —C(O)R_{C-6}, —SOR_{C-6, 20}-SO₂RR_{C-6}, —C(O)N(R_{C-6})₂, —NR_{C-6}C(O)R_{C-6}, —S(O)₂N(R_{C-6})₂, or —NR_{C-6}S(O)₂R_{C-6};

Each R_{C-6} is independently H, alkyl, cycloalkyl, heterocycloalkyl, haloalkyl, halocycloalkyl, or haloheterocycloalkyl;

wherein W is (D):

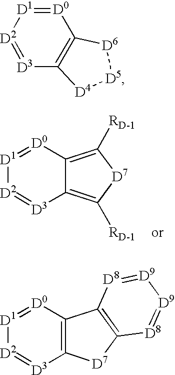

provided that the bond between the —C(═X)— group and the W group may be attached at any available carbon atom within the D group as provided in R_{D-1}, R_{D-3}, and R_{D-4;}

D⁰, D¹, D², and D³ are N or C(R_{D-1}) provided that up to one of D⁰, D¹, D², or D³ is N and the others are C(R_{D-1}), further provided that when C(X) is attached at D² and D⁰ or D¹ is N, D³ is C(H), and further provided that there is only one attachment to C(X);

D⁴—D⁵—D⁶ is selected from N(R_{D-2})—C(R_{D-3})═C(R_{D-3}), N═C(R_{D-3})—C(R_{D-4})₂, C(R_{D-3})═C(R_{D-3})—N(R_{D-2}), C(R_{D-3})₂—N(R_{D-2})—C(R_{D-3})₂, C(R_{D-4})₂—C(R_{D-3})═N, N(R_{D-2})—C(R_{D-3})₂—C(R_{D-3})₂, C(R_{D-3})₂—C(R_{D-3})₂—N(R_{D-2}), O—C(R_{D-3})═C(R_{D-3}), O—C(R_{D-3})₂—C(R_{D-3})₂, C(R_{D-3})₂—O—C(R_{D-3})₂, C(R_{D-3})═C(R_{D-3})—O, C(R_{D-3})₂—C(R_{D-3})₂—O, S—C(R_{D-3})═C(R_{D-3}), S—C(R_{D-3})₂—C(R_{D-3})₂, C(R_{D-3})₂—S—C(R_{D-3})₂, C(R_{D-3})═C(R_{D-3})—S, or C(R_{D-3})₂—C(R_{D-3})₂—S;

provided that when C(X) is attached to W at D² and D⁶ is O, N(R_{D-2}), or S, D⁴—D⁵ is not CH═CH;

and further provided that when C(X) is attached to W at D² and D⁴ is O, N(R_{D-2}), or S, D⁵—D⁶ is not CH═CH;

Each R_{D-1} is independently H, F, Br, I, Cl, —CN, —CF₃, —OR_{D-5}, —SR_{D-5}, —N(R_{D-5})₂, or a bond to C(X) provided that only one R_{D-1} and no R_{D-3} or R_{D-4} is said bond,

Each R_{D-2} is independently H, alkyl, haloalkyl, substituted alkyl, cycloalkyl, halocycloalkyl, substituted cycloalkyl, heterocycloalkyl, haloheterocycloalkyl, substituted heterocycloalkyl, R₅, or R₆;

Each R_{D-3} is independently H, F, Br, Cl, I, alkyl, substituted alkyl, haloalkyl, alkenyl, substituted alkenyl, haloalkenyl, alkynyl, substituted alkynyl, haloalkynyl, heterocycloalkyl, substituted heterocycloalkyl, lactam heterocycloalkyl, —CN, —NO₂, —OR_{D-10}, —C(O)N(R_{D-11})₂, —NR_{D-10}COR_{D-1}₂, —N(R_{D-10})₂, —SR_{D-10}, —S(O)₂R_{D-10}, —C(O)R_{D-1}₂, —CO₂R_{D-10}, aryl, R₅, R₆, or a bond to C(X) provided that only one R_{D-3} and no R_{D-1} or R_{D-4} is also said bond;

Each R_{D-4} is independently H, F, Br, Cl, I, alkyl, substituted alkyl, haloalkyl, alkenyl, substituted alkenyl, haloalkenyl, alkynyl, substituted alkynyl, haloalkynyl, heterocycloalkyl, substituted heterocycloalkyl, lactam heterocycloalkyl, —CN, —NO₂, —OR_{D-10}, —C(O)N(R_{D-11})₂, —NR_{D-10}COR_{D-12}, —N(R_{D-11})₂, —SR_{D-10}, —CO₂R_{D-10}, aryl, R₅, Rr, or a bond to C(X) provided that only one R_{D-4} and no R_{D-1} or R_{D-3} is also said bond;

Each R_{D-5} is independently H, C₁₋₃ alkyl, or C₂₋₄ alkenyl;

D⁷ is O, S, or N(R_{D-2});

D⁸ and D⁹ are C(R_{D-1}), provided that when C(X) is attached at a D⁹, each D⁸ is CH;

Each R_{D-10} is H, alkyl, cycloalkyl, haloalkyl, substituted phenyl, or substituted naphthyl;

Each R_{D-11} is independently H, alkyl, cycloalkyl, heterocycloalkyl, alkyl substituted with 1 substituent selected from R₁₃, cycloalkyl substituted with I substituent selected from R₁₃, heterocycloalkyl substituted with 1 substituent selected from R₁₃, haloalkyl, halocycloalkyl, haloheterocycloalkyl, phenyl, or substituted phenyl;

R_{D-12} is H, alkyl, substituted alkyl, cycloalkyl, haloalkyl, heterocycloalkyl, substituted heterocycloalkyl, substituted phenyl, or substituted naphthyl;

wherein W is (E):

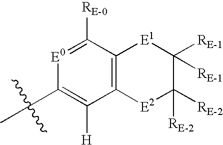

E⁰ is CH or N;

R_{E-0} is H, F, Cl, Br, I, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, aryl, R₅, R₆, —OR_{E-3}, —OR_{E-4}, —SR_{E-3}, —SR_{E-5}, —N(R_{E-3})₂, —NR_{E-3}R_{E-6}, —N(R_{E-6})₂, —C(O)R_{E-3}, —CN, —C(O)N(R_{E-3})₂, —NR_{E-3}C(O)R_{E-3}, —S(O)R_{E-3}, —S(O)R_{E-5}, —OS(O)₂R_{E-3}, —NR_{E-3}S(O)₂R_{E-3}, —NO₂, or —N(H)C(O)N(H)R_{E-3};

E¹ is O, CR_{E-1-1}, or C(R_{E-1-1})₂, provided that when E¹ is CR_{E-1-1}, one R_{E-1} is a bond to CR_{E-1-1}, and further provided that at least one of E¹ or E² is O;

Each R_{E-1-1} is independently H, F, Br, Cl, CN, alkyl, haloalkyl, substituted alkyl, alkynyl, cycloalkyl, —ORE, or —N(RE)₂, provided that at least one R_{E-1-1} is H when E¹ is C(R_{E-1-1})₂;

Each R_{E-1} is independently H, alkyl, substituted alkyl, haloalkyl, cycloalkyl, heterocycloalkyl, or a bond to E¹ provided that E¹ is CR_{E-1-1;}

E² is O, CR_{E-2-2}, or C(R_{E-2-2})₂, provided that when E² is CR_{E-2-2}, one R_{E-2} is a bond to CR_{E-2-2}, and further provided that at least one of E¹ or E² is O;

Each R_{E-2-2} is independently H, F, Br, Cl, CN, alkyl, haloalkyl, substituted alkyl, alkynyl, cycloalkyl, —OR_{E}, or —N(R_{E})₂, provided that at least one R_{E-2-2} is H when E² is C(R_{E-2-2})₂;

Each R_{E-2} is independently H, alkyl, substituted alkyl, haloalkyl, cycloalkyl, heterocycloalkyl, or a bond to E² provided that E² is CR_{E-2-2;}

Each R_{E} is independently H, alkyl, cycloalkyl, heterocycloalkyl, haloalkyl, halocycloalkyl, or haloheterocycloalkyl;

Each R_{E-3} is independently H, alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or phenyl having 1 substituent selected from R₉ and further having 0-3 substituents independently selected from F, Cl, Br, or I or substituted phenyl;

R_{E-4} is H, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;

Each R_{E-5} is independently H, haloalkyl, substituted alkyl, cycloalkyl, halocycloalkyl, substituted cycloalkyl, heterocycloalkyl, haloheterocycloalkyl, substituted heterocycloalkyl, R₅, or R₆;

Each R_{E-6} is independently alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or phenyl having 1 substituent selected from R₉ and further having 0-3 substituents independently selected from F, Cl, Br, or

wherein W is (F):

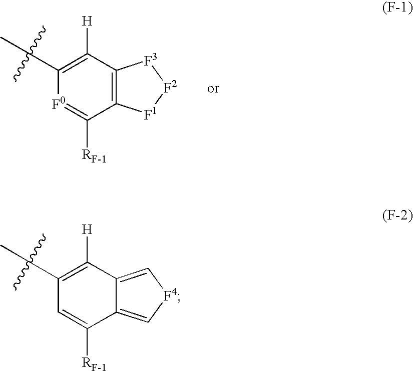

F⁰ is C(H), wherein F¹—F²—F³ is selected from O—C(R_{F-2})═N, O—C(R_{F-3})(R_{F-2})—N(R_{F-4}), O—C(R_{F-3})(R_{F-2})—S, O—N═C(R_{F-3}), O—C(R_{F-2})(R_{F-5})—O, O—C(R_{F-2})(R_{F-3})—O, S—C(R_{F-2})═N, S—C(R_{F-3})(R_{F-2})—N(R_{F-4}), S—N═C(R_{F-3}), N═C(R_{F-2})—O, N═C(R_{F-2})—S, N═C(R_{F-2})—N(R_{F-4}), N(R_{F-4})—N═C(R_{F-3}), N(R_{F-4})—C(R_{F-3})(R_{F-2})—O, N(R_{F-4})—C(R_{F-3})(R_{F-2})—S, N(R_{F-4})—C(R_{F-3})(R_{F-2})—N(R_{F-4}), C(R_{F-3})₂—O—N(R_{F-4}), C(R_{F-3})₂—N(R_{F-4})—O, C(R_{F-3})₂—N(R_{F-4})—S, C(R_{F-3})═N—O, C(R_{F-3})═N—S, C(R_{F-3})═N—N(R_{F-4}), C(R_{F-3})(R_{F-6})—C(R_{F-2})(R_{F-6})—C(R_{F-3})(R_{F-6}), or C(R_{F-3})₂—C(R_{F-2})(R_{F-3})—C(R_{F-3})₂; or

F⁰ is N, wherein F¹—F²—F³ is selected from O—C(R_{F-2})═N, O—C(R_{F-3})(R_{F-2})—N(R_{F-4}), O—C(R_{F-3})(R_{F-2})—S, O—N═C(R_{F-3}) O—C(R_{F-2})(R_{F-3})—O. S—C(R_{F-2})═N, S—C(R_{F-3})(R_{F-2})—N(R_{F-4}), S—N═C(R_{F-3}), N═C(R_{F-2})—O, N═C(R_{F-2})—S, N═C(R_{F-2})—N(R_{F-4}), N(R_{F-4})—N═C(R_{F-3}), N(R_{F-4})—C(R_{F-3})(R_{F-2})—O, N(R_{F-4})—C(R_{F-3})(R_{F-2})—S, N(R_{F-4})—C(R_{F-3})(R_{F-2})—N(R_{F-4}), C(R_{F-3})₂—O—N(R_{F-4}), C(R_{F-3})₂—N(R_{F-4})—O, C(R_{F-3})₂—N(R_{F-4})—S, C(R_{F-3})═N—O, C(R_{F-3})═N—S, C(R_{F-3})═N—N(R_{F-4}), C(R_{F-3})=C(R_{F-2})—C(R_{F-3})₂, or C(R_{F-3})₂—C(R_{F-2})(R_{F-3})—C(R_{F-3})₂;

F⁴ is N(R_{F-7}), O, or S;

R_{F-1} is H, F, Cl, Br, I, —CN, —CF₃, —OR_{F-8}, —SR_{F-8}, or —N(R_{F-8})₂;

R_{F-2} is H, F, alkyl, haloalkyl, substituted alkyl, lactam heterocycloalkyl, phenoxy, substituted phenoxy, R₅, Rr, —N(R_{F-4})-aryl, —N(R_{F-4})-substituted phenyl, —N(R_{F-4})-substituted naphthyl, —O-substituted phenyl, —O-substituted naphthyl, —S-substituted phenyl, —S-substituted naphthyl, or alkyl substituted on the ω carbon with R_{F-9} where said ω carbon is determined by counting the longest carbon chain of the alkyl moiety with the C-1 carbon being the carbon attached to W and the ω carbon being the carbon furthest, e.g., separated by the greatest number of carbon atoms in the chain, from said C-1 carbon;

R_{F-3} is H, F, Br, Cl, I, alkyl, substituted alkyl, haloalkyl, alkenyl, substituted alkenyl, haloalkenyl, alkynyl, substituted alkynyl, haloalkynyl, heterocycloalkyl, substituted heterocycloalkyl, lactam heterocycloalkyl, —CN, —NO₂, —OR_{F-8}, —C(O)N(R_{F-8})₂, —NHR_{F-8}, —NR_{F-8}COR_{F-8}, —N(R_{F-8})₂, —SR_{F-8}, —C(O)R_{F-8}, —CO₂R_{F-8}, aryl, R₅, or R₆;

R_{F-4} is H, or alkyl;

Each R_{F-5} is independently F, Br, Cl, I, alkyl, substituted alkyl, haloalkyl, alkenyl, substituted alkenyl, haloalkenyl, alkynyl, substituted alkynyl, haloalkynyl, —CN, —CF₃, —OR_{F-8}, —C(O)NH₂, —NHR_{F-8}, —SR_{F-8}, —CO₂R_{F-8}, aryl, phenoxy, substituted phenoxy, R₅, R₆, —N(R_{F-4})-aryl, or —O-substituted aryl;

One of R_{F-6} is H, alkyl, substituted alkyl, haloalkyl, alkenyl, substituted alkenyl, haloalkenyl, alkynyl, substituted alkynyl, haloalkynyl, —CN, F, Br, Cl, I, —OR_{F-8}, —C(O)NH₂, —NHR_{F-8}, —SR_{F-8}, —CO₂R_{F-8}, aryl, R₅, or R₆, and each of the other R_{F-6} is independently selected from alkyl, substituted alkyl, haloalkyl, alkenyl, substituted alkenyl, haloalkenyl, alkynyl, substituted alkynyl, haloalkynyl, —CN, F, Br, Cl, I, —OR_{F-8}, —C(O)NH₂, —NHR_{F-8}, —SR_{F-8}, —CO₂R_{F-8}, aryl, R₅, or R₆;

R_{F-7} is H, alkyl, haloalkyl, substituted alkyl, cycloalkyl, halocycloalkyl, substituted cycloalkyl, phenyl, or phenyl having 1 substituent selected from R₉ and further having 0-3 substituents independently selected from F, Cl, Br, or I;

R_{F-8} is H, alkyl, substituted alkyl, cycloalkyl, haloalkyl, heterocycloalkyl, substituted heterocycloalkyl, substituted phenyl, or substituted naphthyl;

R_{F-9} is aryl, R₅, or R₆;

wherein W is (G):

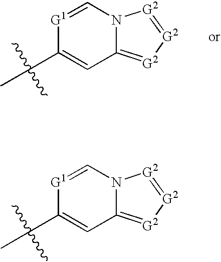

G¹ is N or CH;

Each G² is N or C(R_{G-1}), provided that no more than one G is N, and further provided that when G² adjacent to the bridge N is C(R_{G-1}) and the other G² are CH, that R_{G-1} is other than H, F, Cl, I, alkyl, substituted alkyl or alkynyl;

Each R_{G-1} is independently H, alkyl, substituted alkyl, haloalkyl, alkenyl, substituted alkenyl, haloalkenyl, alkynyl, substituted alkynyl, haloalkynyl, —CN, —NO₂, F, Br, Cl, I, —C(O)N(R_{G-3})₂, —N(R_{G-3})₂, —SR_{G-6}, —S(O)₂R_{G-6}, —OR_{G-6}, —C(O)R_{G-6}, —CO₂R_{G-6}, aryl, R₅, R₆, or two R_{G-1} on adjacent carbon atoms may combine for W to be a 6-5-6 fused-tricyclic-heteroaromatic-ring system optionally substituted on the newly formed ring where valency allows with 1-2 substitutents independently selected from F, Cl, Br, I, and R_{G-2};

R_{G-2} is alkyl, alkenyl, alkynyl, cycloalkyl, heterocycloalkyl, haloalkyl, haloalkenyl, haloalkynyl, halocycloalkyl, haloheterocycloalkyl, —OR_{G-8}, —SR_{G-8}, —S(O)₂R_{G-8}, —S(O)R_{G-8}, —OS(O)₂R_{G-8}, —N(R_{G-8})₂, —C(O)R_{G-8}, —C(S)R_{G-8}, —C(O)OR_{G-8}, —CN, —C(O)N(R_{G-8})₂, —NR_{G-8}C(O)R_{G-8}, —S(O)₂N(R_{G-8})₂, —NR_{G-8}S(O)₂R_{G-8}, —NO₂, —N(R_{G-8})C(O)N(R_{G-8})₂, substituted alkyl, substituted alkenyl, substituted alkynyl, substituted cycloalkyl, substituted heterocycloalkyl, lactam heterocycloalkyl, phenyl, phenyl having 0-4 substituents independently selected from F, Cl, Br, I and R_{G-7}, naphthyl, or naphthyl having 0-4 substituents independently selected from F, Cl, Br, I, or R_{G-7;}

Each R_{G-3} is independently H, alkyl, cycloalkyl, heterocycloalkyl, alkyl substituted with 1 substituent selected from R_{G-4}, cycloalkyl substituted with 1 substituent selected from R_{G-4}, heterocycloalkyl substituted with 1 substituent selected from R_{G-4}, haloalkyl, halocycloalkyl, haloheterocycloalkyl, phenyl, or substituted phenyl;

R_{G-4} is —OR_{G-5}, —SR_{G-5}, —N(R_{G-5})₂, —C(O)R_{G-5}, —SOR_{G-5}, —SO₂R_{G-5}, —C(O)N(R_{G-5})₂, —CN, —CF₃, —NR_{G-5}C(O)R_{G-5}, —S(O)₂N(R_{G-5})₂, —NR_{G-5}S(O)₂R_{G-5}, or —NO₂;

Each R_{G-5} is independently H, alkyl, cycloalkyl, heterocycloalkyl, haloalkyl, halocycloalkyl, or haloheterocycloalkyl;

R_{G-6} is H, alkyl, haloalkyl, substituted alkyl, cycloalkyl, halocycloalkyl, substituted cycloalkyl, phenyl, or phenyl having 0-4 substituents independently selected from F, Cl, Br, I, and R_{G-7;}

R_{G-7} is alkyl, substituted alkyl, haloalkyl, —OR_{G-5}, —CN, —NO₂, —N(R_{G-3})₂;

Each R_{G-8} is independently H, alkyl, haloalkyl, substituted alkyl, cycloalkyl, halocycloalkyl, substituted cycloalkyl, heterocycloalkyl, haloheterocycloalkyl, substituted heterocycloalkyl, phenyl, or phenyl substituted with 0-4 independently selected from F, Cl, Br, I, or R_{G-7;}

wherein W is (H)

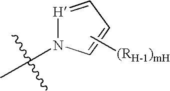

H′ is N or CH;

Each R_{H-1} is independently F, Cl, Br, I, —CN, —NO₂, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocyloalkyl, substituted heterocycloalkyl, lactam heterocyclcoalkyl, aryl, R₅, R₆, —OR_{H-3}, —SR_{H-3}, —SOR_{H-3}, —SO₂R_{H-3}, —SCN, —S(O)N(R_{H-3})₂, —S(O)₂N(R_{H-3})₂, —C(O)R_{H-3}, —C(O)₂R_{H-3}, —C(O)N(R_{H-3})₂, —C(R_{H-3})═N—OR_{H-3}, —NC(O)R_{H-3}, —NC(O)R_{H-3}, —NC(O)R_{H-3}, —N(R_{H-3})₂, —NR_{H-3}C(O)R_{H-3}, —NR_{H-3}S(O)₂R_{H-3}, or two R_{H-1} on adjacent carbon atoms may fuse to form a 6-membered ring to give a 5-6 fused, bicyclic moiety where the 6-membered ring is optionally substituted with 1-3 substitutents selected from R_{H-2;}

m_{H} is 0, 1, or 2;

R_{H-2} is alkyl, alkenyl, alkynyl, cycloalkyl, heterocycloalkyl, haloalkyl, haloalkenyl, haloalkynyl, halocycloalkyl, haloheterocycloalkyl, —OR_{H-3}, —SR_{H-3}, —S(O)₂R_{H-3}, —S(O)R_{H-3}, —OS(O)₂R_{H-3}, —N(R_{H-3})₂, —C(O)R_{H-3}, —C(S)R_{H-3}, —C(O)OR_{H-3}, —CN, —C(O)N(R_{H-3})₂, —NR_{H-3}C(O)R_{H-3}, —S(O)₂N(R_{H-3})₂, —NR_{H-3}S(O)₂R_{H-3}, —NO₂, —N(R_{H-3})C(O)N(R_{H-3})₂, substituted alkyl, substituted alkenyl, substituted alkynyl, substituted cycloalkyl, substituted heterocycloalkyl, lactam heterocycloalkyl, phenyl, phenyl having 0-4 substituents independently selected from F, Cl, Br, I and R₇, naphthyl, naphthyl having 0-4 substituents independently selected from F, Cl, Br, I, or R₇, or two R_{H-2} on adjacent carbon atoms may combine to form a three-ring-fused-5-6-6 system optionally substituted with up to 3 substituents independently selected from Br, Cl, F, I, —CN, —NO₂, —CF₃, —N(R_{H-3})₂, —N(R_{H-3})C(O)R_{H-3}, alkyl, alkenyl, and alkynyl;

Each R_{H-3} is independently H, alkyl, haloalkyl, substituted alkyl, cycloalkyl, halocycloalkyl, substituted cycloalkyl, heterocycloalkyl, haloheterocycloalkyl, substituted heterocycloalkyl, phenyl, or phenyl substituted with 0-4 independently selected from F, Cl, Br, I, or R₇;

or pharmaceutical composition, pharmaceutically acceptable salt, racemic mixture, or pure enantiomer thereof; and

provided that the compound of Formula I includes at least one isotopic label.

Examples of isotopic atoms that can be incorporated into compounds of the invention include, but are not limited to, isotopes of hydrogen, carbon, nitrogen, oxygen, phosphorous, fluorine, iodine, and chlorine, such as ²H, ³H, ¹¹C, ¹³C, ¹⁴C, ¹⁸F, ¹⁹F, ¹²³I, ¹²⁵I, and ^{99m}Tc.

The invention also provides a method of utilizing an isotopically labeled compound of formula I to perform diagnostic screening, such as PET, SPECT, and NMR spectroscopy. The compounds of the present invention are useful in diagnostic analysis of a disease or condition as described herein in a mammal. The present invention further provides compounds that are useful in diagnostic analysis of a disease or condition in a mammal, including where the alpha 7 nAChR is implicated and modulation of the alpha 7 nAChR is desired or where the alpha 7 nAChR is implicated and modulation of the alpha 7 nAChR is desired.

In accordance with the present invention, the compounds that are administered are detected using methods such as PET and SPECT. The present invention allows one skilled in the art of the use of diagnostic tools, such as PET and SPECT, to diagnose a wide variety of conditions and disorders, including conditions and disorders associated with dysfunction of the central and autonomic nervous system. The present invention is useful in the diagnosis of a wide variety of disease and disorders where the alpha 7 nAChR is implicated, including cognitive and attention deficit symptoms of Alzheimer's, neurodegeneration associated with diseases such as Alzheimer's disease, pre-senile dementia (mild cognitive impairment), senile dementia, schizophrenia, psychosis, attention deficit disorder, attention deficit hyperactivity disorder, depression, anxiety, general anxiety disorder, post traumatic stress disorder, mood and affective disorders, amyotrophic lateral sclerosis, borderline personality disorder, traumatic brain injury, behavioral and cognitive problems in general and associated with brain tumors, AIDS dementia complex, dementia associated with Down's syndrome, dementia associated with Lewy Bodies, Huntington's disease, Parkinson's disease, tardive dyskinesia, Pick's disease, dysregulation of food intake including bulemia and anorexia nervosa, withdrawal symptoms associated with smoking cessation and dependant drug cessation, Gilles de la Tourette's Syndrome, age-related macular degeneration, glaucoma, neurodegeneration associated with glaucoma, diabetic retinopathy, or symptoms associated with pain.

Abbreviations which are well known to one of ordinary skill in the art may be used (e.g., “Ph” for phenyl, “Me” for methyl, “Et” for ethyl, “h” or “hr” for hour or hours, “min” for minute or minutes, and “rt” for room temperature).

All temperatures are in degrees Centigrade.

Room temperature is within the range of 15-25 degrees Celsius.

AChR refers to acetylcholine receptor.

nAChR refers to nicotinic acetylcholine receptor.

Pre-senile dementia is also known as mild cognitive impairment.

5HT₃R refers to the serotonin-type 3 receptor.

α-btx refers to α-bungarotoxin.

FLIPR refers to a device marketed by Molecular Devices, Inc. designed to precisely measure cellular fluorescence in a high throughput whole-cell assay. (Schroeder et. al., J. Biomolecular Screening, 1(2), p 75-80, 1996).

TLC refers to thin-layer chromatography.

HPLC refers to high pressure liquid chromatography.

MeOH refers to methanol.

EtOH refers to ethanol.

IPA refers to isopropyl alcohol.

THF refers to tetrahydrofuran.

DMSO refers to dimethylsulfoxide.

DMF refers to N,N-dimethylformamide.

EtOAc refers to ethyl acetate.

TMS refers to tetramethylsilane.

TEA refers to triethylamine.

DIEA refers to N,N-diisopropylethylamine.

MLA refers to methyllycaconitine.

Ether refers to diethyl ether.

HATU refers to 0-(7-azabenzotriazol-1-yl)-N,N,N′,N′-tetramethyluronium hexafluorophosphate.

CDI refers to carbonyl diimidazole.

NMO refers to N-methylmorpholine-N-oxide.

TPAP refers to tetrapropylammonium perruthenate.

Na₂SO₄ refers to sodium sulfate.

K₂CO₃ refers to potassium carbonate.

MgSO₄ refers to magnesium sulfate.

When Na₂SO₄, K₂CO₃, or MgSO₄ is used as a drying agent, it is anhydrous.

Halogen is F, Cl, Br, or I.

The carbon atom content of various hydrocarbon-containing moieties is indicated by a prefix designating the minimum and maximum number of carbon atoms in the moiety, i.e., the prefix Cᵢ₋ⱼ indicates a moiety of the integer ‘i” to the integer “j” carbon atoms, inclusive. Thus, for example, C₁₋₆ alkyl refers to alkyl of one to six carbon atoms.

Non-inclusive examples of compounds that fall within the definition of R₅ and R₆ include, but are not limited to, thienyl, benzothienyl, pyridyl, thiazolyl, quinolyl, pyrazinyl, pyrimidyl, imidazolyl, furanyl, benzofuranyl, benzothiazolyl, isothiazolyl, benzisothiazolyl, benzisoxazolyl, benzimidazolyl, indolyl, benzoxazolyl, pyrazolyl, triazolyl, tetrazolyl, isoxazolyl, oxazolyl, pyrrolyl, isoquinolinyl, cinnolinyl, indazolyl, indolizinyl, phthalazinyl, pydridazinyl, triazinyl, isoindolyl, purinyl, oxadiazolyl, furazanyl, benzofurazanyl, benzothiophenyl, benzothiazolyl, quinazolinyl, quinoxalinyl, naphthridinyl, and furopyridinyl. One of ordinary skill in the art will recognize whether the moiety would fall under R₅ or R₆ by comparing the moiety with the definitions of R₅ and R₆.

Non-inclusive examples of heterocycloalkyl include, but are not limited to, tetrahydrofurano, tetrahydropyrano, morpholino, pyrrolidino, piperidino, piperazine, azetidino, azetidinono, oxindolo, dihydroimidazolo, and pyrrolidinono

Some of the amines described herein require the use of an amine-protecting group to ensure functionalization of the desired nitrogen. One of ordinary skill in the art would appreciate where, within the synthetic scheme, to use said protecting group. Amino protecting group includes, but is not limited to, carbobenzyloxy (CBz), tert butoxy carbonyl (BOC) and the like. Examples of other suitable amino protecting groups are known to person skilled in the art and can be found in “Protective Groups in Organic synthesis,” 3rd Edition, authored by Theodora Greene and Peter Wuts.

Alkyl substituted on an ω carbon with R_{A-7} is determined by counting the longest carbon chain of the alkyl moiety with the C-1 carbon being the carbon attached to the W moiety and the ω carbon being the carbon furthest, e.g., separated by the greatest number of carbon atoms in the chain, from said C-1 carbon. Therefore, when determining the c) carbon, the C-1 carbon will be the carbon attached, as valency allows, to the W moiety and the co carbon will be the carbon furthest from said C-1 carbon.

The core molecule is Azabicyclo-N(H)—C(═O)—:

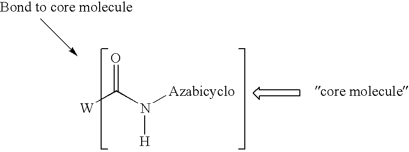

Mammal denotes a human being, and other mammals and animals, such as food animals (e.g., cows, pigs, sheep, goats, deer, poultry, etc.), companion animals (e.g., dogs, cats, horses, birds, and fish), or other mammals.

Brine refers to an aqueous saturated sodium chloride solution.

Equ means molar equivalents.

IR refers to infrared spectroscopy.

Lv refers to leaving groups within a molecule, including Cl, OH, or mixed anhydride.

NMR refers to nuclear (proton) magnetic resonance spectroscopy, chemical shifts are reported in ppm (δ) downfield from TMS.

MS refers to mass spectrometry expressed as m/e or mass/charge unit. HRMS refers to high resolution mass spectrometry expressed as m/e or mass/charge unit. [M+H]⁺ refers to an ion composed of the parent plus a proton. [M−H]⁻ refers to an ion composed of the parent minus a proton. [M+Na]⁺ refers to an ion composed of the parent plus a sodium ion. [M+K]⁺ refers to an ion composed of the parent plus a potassium ion. EI refers to electron impact. ESI refers to electrospray ionization. CI refers to chemical ionization. FAB refers to fast atom bombardment.

Compounds of the present invention may be in the form of pharmaceutically acceptable salts. The term “pharmaceutically acceptable salts” refers to salts prepared from pharmaceutically acceptable non-toxic bases including inorganic bases and organic bases, and salts prepared from inorganic acids, and organic acids. Salts derived from inorganic bases include aluminum, ammonium, calcium, ferric, ferrous, lithium, magnesium, potassium, sodium, zinc, and the like. Salts derived from pharmaceutically acceptable organic non-toxic bases include salts of primary, secondary, and tertiary amines, substituted amines including naturally occurring substituted amines, cyclic amines, such as arginine, betaine, caffeine, choline, N,N-dibenzylethylenediamine, diethylamine, 2-diethylaminoethanol, 2-dimethylamino-ethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and the like. Salts derived from inorganic acids include salts of hydrochloric acid, hydrobromic acid, hydroiodic acid, sulfuric acid, phosphoric acid, phosphorous acid and the like. Salts derived from pharmaceutically acceptable organic non-toxic acids include salts of C₁₋₆ alkyl carboxylic acids, di-carboxylic acids, and tri-carboxylic acids such as acetic acid, propionic acid, fumaric acid, succinic acid, tartaric acid, maleic acid, adipic acid, and citric acid, and aryl and alkyl sulfonic acids such as toluene sulfonic acids and the like.

In addition to the compound(s) of Formula I, the composition for diagnostic use may also comprise one or more non-toxic, pharmaceutically acceptable carrier materials or excipients. A generally recognized compendium of such methods and ingredients is Remington's Pharmaceutical Sciences by E. W. Martin (Mark Publ. Co., 15th Ed., 1975). The term “carrier” material or “excipient” herein means any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, and then tableted or encapsulated for convenient administration. Such capsules or tablets may contain a controlled-release formulation as may be provided in a dispersion of active compound in hydroxypropyl-methyl cellulose, or other methods known to those skilled in the art. For oral administration, the pharmaceutical composition may be in the form of, for example, a tablet, capsule, suspension or liquid. If desired, other active ingredients may be included in the composition.

In addition to the oral dosing, noted above, the compositions of the present invention may be administered by any suitable route, e.g., parenterally, bucal, intravaginal, and rectal, in the form of a pharmaceutical composition adapted to such a route, and in a dose effective for the treatment intended. Such routes of administration are well known to those skilled in the art. The compositions may, for example, be administered parenterally, e.g., intravascularly, intraperitoneally, subcutaneously, or intramuscularly. For parenteral administration, saline solution, dextrose solution, or water may be used as a suitable carrier. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, EtOH, corn oil, cottonseed oil, peanut oil, sesame oil, benzyl alcohol, sodium chloride, and/or various buffers. Other adjuvants and modes of administration are well and widely known in the pharmaceutical art. The preferred route of administration is by intravenous route.

The serotonin type 3 receptor (5HT₃R) is a member of a superfamily of ligand-gated ion channels, which includes the muscle and neuronal nAChR, the glycine receptor, and the γ-aminobutyric acid type A receptor. Like the other members of this receptor superfamily, the 5HT₃R exhibits a large degree of sequence homology with α7 nAChR but functionally the two ligand-gated ion channels are very different. For example, α7 nAChR is rapidly inactivated, is highly permeable to calcium and is activated by acetylcholine and nicotine. On the other hand, 5HT₃R is inactivated slowly, is relatively impermeable to calcium and is activated by serotonin. These experiments suggest that the α7 nAChR and 5HT₃R proteins have some degree of homology, but function very differently. Indeed the pharmacology of the channels is very different. For example, Ondansetron, a highly selective 5HT₃R antagonist, has little activity at the α7 nAChR. The converse is also true. For example, GTS-21, a highly selective α7 nAChR agonist, has little activity at the 5HT₃R.

α7 nAChR is a ligand-gated Ca⁺⁺ channel formed by a homopentamer of α7 subunits. Previous studies have established that α-bungarotoxin (α-btx) binds selectively to this homopetameric, α7 nAChR subtype, and that α7 nAChR has a high affinity binding site for both α-btx and methyllycaconitine (MLA). α7 nAChR is expressed at high levels in the hippocampus, ventral tegmental area and ascending cholinergic projections from nucleus basilis to thalamocortical areas. α7 nAChR agonists increase neurotransmitter release, and increase cognition, arousal, attention, learning and memory.

Data from human and animal pharmacological studies establish that nicotinic cholinergic neuronal pathways control many important aspects of cognitive function including attention, learning and memory (Levin, E. D., Psychopharmacology, 108:417-31, 1992; Levin, E. D. and Simon B. B., Psychopharmacology, 138:217-30, 1998). For example, it is well known that nicotine increases cognition and attention in humans. ABT-418, a compound that activates α4β2 and α7 nAChR, improves cognition and attention in clinical trials of Alzheimer's disease and attention-deficit disorders (Potter, A. et. al., Psychopharmacology (Berl)., 142(4):334-42, March 1999; Wilens, T. E. et. al., Am. J. Psychiatry, 156(12):1931-7, December 1999). It is also clear that nicotine and selective but weak α7 nAChR agonists increase cognition and attention in rodents and non-human primates.

Selective α7 nAChR agonists may be found using a functional assay on FLIPR (see WO 00/73431 A2). FLIPR is designed to read the fluorescent signal from each well of a 96 or 384 well plate as fast as twice a second for up to 30 minutes. This assay may be used to accurately measure the functional pharmacology of α7 nAChR and 5HT₃R. To conduct such an assay, one uses cell lines that expressed functional forms of the α7 nAChR using the 0:7/5-HT₃ channel as the drug target and cell lines that expressed functional 5HT₃R. In both cases, the ligand-gated ion channel was expressed in SH-EP1 cells. Both ion channels can produce robust signal in the FLIPR assay.

The present invention is useful in the diagnosis of a wide variety of disease and disorders where the alpha 7 nAChR is implicated, including any one or more of the following: cognitive and attention deficit symptoms of Alzheimer's, neurodegeneration associated with diseases such as Alzheimer's disease, pre-senile dementia (mild cognitive impairment), senile dementia, schizophrenia, psychosis, attention deficit disorder, attention deficit hyperactivity disorder, depression, anxiety, general anxiety disorder, post traumatic stress disorder, mood and affective disorders, amyotrophic lateral sclerosis, borderline personality disorder, traumatic brain injury, behavioral and cognitive problems in general and associated with brain tumors, AIDS dementia complex, dementia associated with Down's syndrome, dementia associated with Lewy Bodies, Huntington's disease, Parkinson's disease, tardive dyskinesia, Pick's disease, dysregulation of food intake including bulemia and anorexia nervosa, withdrawal symptoms associated with smoking cessation and dependant drug cessation, Gilles de la Tourette's Syndrome, age-related macular degeneration, glaucoma, neurodegeneration associated with glaucoma, diabetic retinopathy, or symptoms associated with pain.

The key step in the preparation of this class of PET ligands is the coupling of the Azabicyclo moiety with the requisite aryl or heteroaryl halogen or triflate, via a palladium-mediated reaction with [¹¹C] carbon monoxide. The method utilized has been described in detail by T. Kihlberg and B. Langstrom in J. Org. Chem., 1999, 64, 9201-5. In general, the ¹¹C-labeled amides are synthesized at high pressures in a micro autoclave using a solution of W-Lv, palladium tetrakis triphenylphosphine, an azabicyclic amine, [¹¹C] carbon monoxide in 1,4-dioxane at 130-150° C. The compounds where Lv is I or OTf are the generally preferred compounds for the preparation of this class of molecules. One of ordinary skill in the art will recognize that the requisite intermediates W-Lv are either commercially available or can be prepared using procedures known in the art.

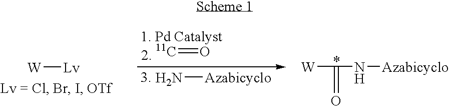

Preparation of the PET ligand aryl-furan can proceed via two routes. These preparations exemplifying the use of one heteroaryl are applicable to related heteroaryls. In the first route, the commercially available di-substituted furan is coupled to an aromatic dimethylamino boronate in a Pd-mediated reaction. The product can then be readily converted to the trimethylammonium salt, followed by nucleophilic displacement of the trimethylamino moiety with [¹⁸F]fluoride using high temperatures or microwave irradiation. Similar radiofluorinations of heteroaryl-substituted benzene systems have been reported (McCarthy, T. J., et al. J. Nuc. Med., 2002, 43, 117-124). Ester hydrolysis and HATU coupling with the azabicyclic amine can then proceed as discussed herein.

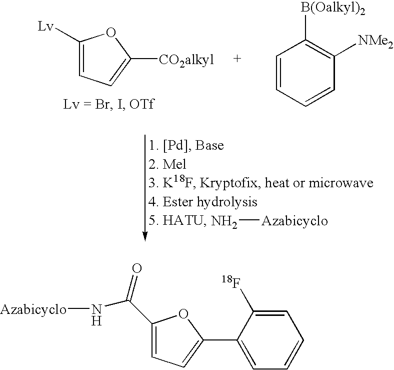

In the second route, the nucleophilic fluorination is performed on the nitro precursor. There are many examples of aromatic nitro-[¹⁸F] substitutions in the literature (see, e.g., Kilbourn, M. R. Fluorine-18 Labeling of Radiopharmaceuticals; National Academy Press: Washington, D.C., 1990; and Attina, M., et al. J. Labelled Comp. Radiopharm., 1983, 20, 501-514). While most of these are performed on “activated” nitroaromatics, examples do exist for relatively unactivated substrates (Tang, G-H., et al. He Huaxue Yu Fangshe Huaxue, 2002, 23, 211-216; Stone-Elander, S., et al. Appl. Rad. Isot., 1993, 44, 889-893; Ding, Y. S., et al. J. Med. Chem., 1991, 34, 767-771; Lemaire, C., et al. J. Nuc. Med., 1990, 31, 1247-¹²⁵I). The chemistry is broadly applicable to other 2-nitrophenyl-substituted heteroaryls.

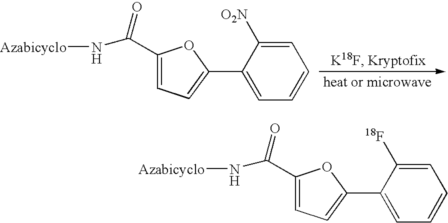

The ⁷⁶Br-labeled thiophenes for PET can be prepared by reacting the trialkylstannyl precursor with the ⁷⁶Br source (typically Na ⁷⁶Br or NH₄⁷⁶ Br). This reaction also employs an oxidizing agent such as chloramine-T, peracetic acid, or H₂O₂. These bromodestannylation conditions are known in the literature (e.g. Yngve, U., et al., J. Labelled Comp. Radiopharm., 1997, 39, 120-121; Strijckmans, V., et al. J. Labelled Comp. Radiopharm., 1997, 39, 339-348; Kassiou, M., et al. J. Labelled Comp. Radiopharm., 2000, 43, 339-346; Kao, C-H., et al. J. Labelled Comp. Radiopharm., 2001, 44, 889-898). The trialkylstannyl precursors can be made by procedures known to those of ordinary skill in the art. This chemistry may be carried out on other trialkylstanyl substituted heteroaryls. Ttrialkylstannyl precursors can be made by those of ordinary skill in the art. See, e.g., Burnett, et al., Bioorg. & Med. Chemc Lett. 12 (2002) 311-314; Li, G. and Bittman, R., Tet. Lett. 41 (2000) 6737-6741.

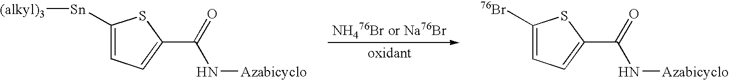

For SPECT, the ¹²³I- or ¹²⁵I-labeled 1H-pyrazolyl ligand intermediate can be prepared in a manner analogous to the ⁷⁶Br-labeled compounds. Once again, the reaction employs the displacement of a trialkylstannyl moiety with *I (available as Na*I, where * is 123 or 125). As in the bromodestannylation, a co-oxidant such as chloramine-T is necessary for the reaction to occur. This method of preparation is standard in the field of radiochemistry (see Seevers, R. H., et al. Chem. Rev., 1982, 82, 575-590 and Baldwin, R. M. Appl. Radiat. Isot-Int. J. Rad. A., 1986, 37, 817-821).

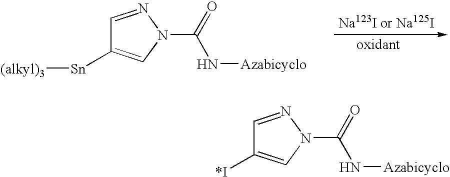

One of ordinary skill in the art will recognize that the methods described for the reaction of the unsubstituted 3-amino-1-azabicyclo[2.2.1]heptane (R₂═H) are equally applicable to substituted compounds (R₂≠H). For where Azabicyclo is II, compounds where R₂ is present can be prepared from appropriately substituted nitro alcohols using procedures described in Tetrahedron (1997), 53, p. 11121 as shown below. Methods to synthesize nitro alcohols are well known in the art (see J. Am. Chem. Soc. (1947), 69, p 2608). The scheme below is a modification of the synthesis of exo-3-amino-1-azabicyclo[2.2.1]heptane as the bis(hydro para-toluenesulfonate) salt, described in detail herein, to show how to obtain these amine precursors. The desired salt can be made using standard procedures.

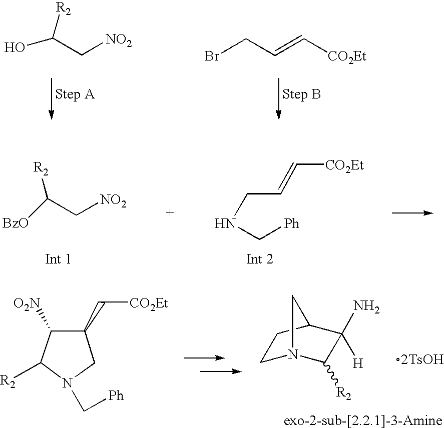

Compounds for Azabicyclo II where R₂ is other than H can also be prepared by modification of intermediates described in the synthesis of exo-3-amino-1-azabicyclo[2.2.1]heptane as the bis(hydro para-toluenesulfonate) salt, described in detail herein. For example, Int 6 can be oxidized to the aldehyde and treated with an organometallic reagent to provide Int 20 using procedures described in Tetrahedron (1999), 55, p 13899. Int 20 can be converted into the amine using methods described for the synthesis of exo-3-amino-1-azabicyclo[2.2.1]heptane as the bis(hydro para-toluenesulfonate) salt. Once the amine is obtained, the desired salt can be made using standard procedures.

The schemes used are for making exo-3-amino-1-azabicyclo[2.2.1]heptane. However, the modifications discussed are applicable to make the endo isomer also.

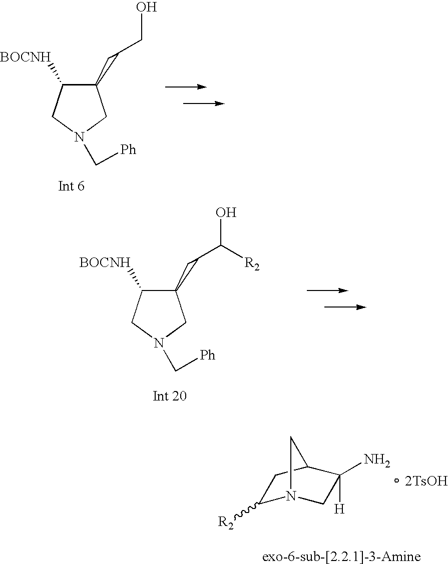

The exo- and endo-1-azabicyclo[3.2.1]octan-3-amines are prepared from 1-azabicyclic[3.2.1]octan-3-one (Thill, B. P., Aaron, H. S., J. Org. Chem., 4376-4380 (1968)) according to the general procedure as discussed in Lewin, A. H., et al., J. Med. Chem., 988-995 (1998).

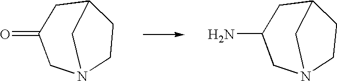

One of ordinary skill in the art will also recognize that the methods described for the reaction of the unsubstituted 1-azabicyclo[3.2.1]octan-3-amine (R₂ is H) are equally applicable to substituted compounds (R₂ present). The R₂ substituent may be introduced as known to one skilled in the art through standard alkylation chemistry. Exposure of 1-azabicyclo[3.2.1]octan-3-one to a hindered base such as LDA (lithium diisopropylamide) in a solvent such as THF or ether between 0° C. to −78° C. followed by the addition of an alkylating agent (R₂Lv, where Lv=Cl, Br, I, OTs, etc.) will, after being allowed to warm to about 0° C. to rt followed by an aqueous workup, provide the desired compound as a mixture of isomers. Chromatographic resolution (flash, HPLC, or chiral HPLC) will provided the desired purified alkylated ketones. From there, formation of the oxime and subsequent reduction will provide the desired endo or exo isomers.

There are various methods for the construction of the optionally substituted 7-azabicyclo[2.2.1]heptane ring system. For example, the independent work of Trudell (R₃═H, Zhang, C., Trudell, M. L., J. Org. Chem., 61, 7189-7191, 1996), and Schultz (R₃=Me, Schultz, A. G., Shen, M. S., Tetrahedron Lett., 22, 3347-3350, 1981) describes the utility of a Diels-Alder approach toward preparing this ring system with functionality suitable for further elaboration to the desired 2-amino-7-aza-bicyclo[2.2.1]heptane (Scheme 2). For instance, Trudell reports (Zhang, C., Trudell, M. L., Tetrahedron, 54, 8349-8354, 1998) that Diels-Alder adduct 1a (where R₄=methylcarbamate, R₃═H, and Lv=Br) could readily be functionalized at C-3 via reaction with organocopper species to introduce the substituent R₂ in 2a,b. Likewise, hydrogenolysis of adduct 1a,b or 2a,b followed by isomerization of the endo products as described by Singh (Singh, S., Basmadjian, G. P., Tetrahedron Lett., 38, 6829-6830, 1997) could provide access to the required exo acid 3a-d. Treatment of 3 with diphenylphosphoryl azide in the presence of a tertiary amine base (e.g., Et₃N) in a suitable solvent such as toluene, followed by warming of the intermediate acylazide in the presence of a suitable alcohol (e.g., benzyl alcohol) would effect the well-known Curtius rearrangement to provide a differentially protected bis carbamate which could be cleaved under typical hydrogenolysis conditions (e.g., 10% Pd/C, EtOH, H₂, ambient to 50 psi) to give the desired amine 4. Alternatively, the differentially protected bis carbamate might provide an attractive point of intervention for the chromatographic resolution of the individual 2-exo isomers prior to cleavage to amine 4.

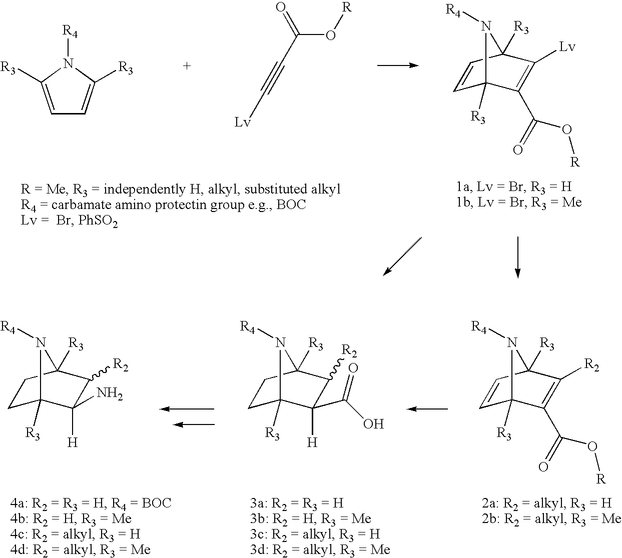

In the case where R₄=tert-butyloxycarbonyl, deprotection of the 7-aza group can be conveniently accomplished under acidic conditions in a suitable solvent such as methanol. After deprotection, the secondary amine may be functionalized with alkyl and substituted alkyl via reductive amination or alkylative procedures.

Preparation of the 2.2.1 Amines:

Synthesis of exo-3-amino-1-azabicyclo[2.2.1 Jheptane as the bis(hydro para-toluenesulfonate) salt:

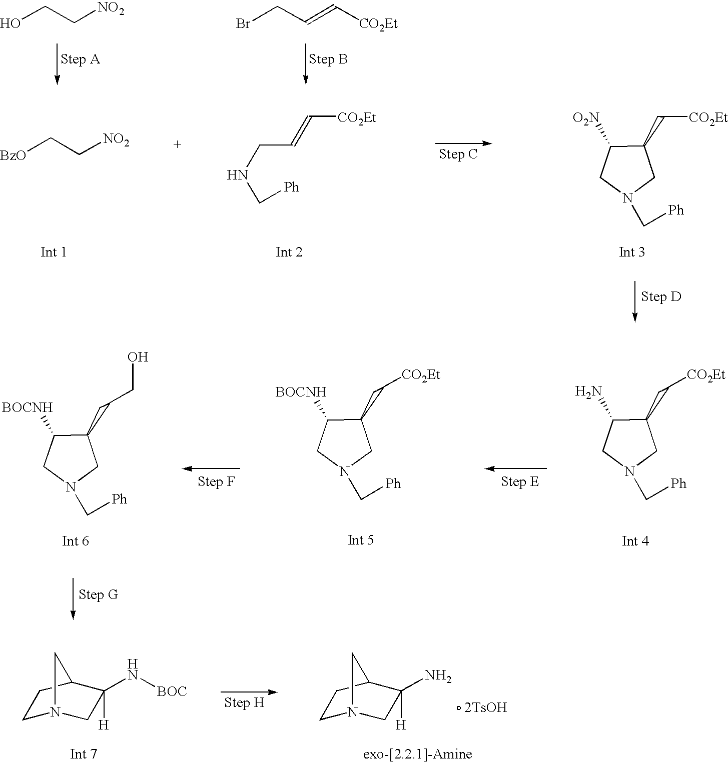

Step A. Preparation of 2-(benzoyloxy)-1-nitroethane (Int 1).

Benzoyl chloride (14.9 mL, 128 mmol) is added to a stirred solution of nitroethanol (9.2 mL, 128 mmol) in dry benzene (120 mL). The solution is refluxed for 24 hr and then concentrated in vacuo. The crude product is purified by flash chromatography on silica gel. Elution with hexanes-EtOAc (80:20) affords Int 1 as a white solid (68% yield): ¹H NMR (CDCl₃) δ 8.0, 7.6, 7.4, 4.9, 4.8.

Step B. Preparation of ethyl E-4-(benzylamino)-2-butenoate (Int 2).

Ethyl E-4-bromo-2-butenoate (10 mL, 56 mmol, tech grade) is added to a stirred solution of benzylamine (16 mL, 146 mmol) in CH₂Cl₂ (200 mL) at rt. The reaction mixture stirs for 15 min, and is diluted with ether (1 L). The mixture is washed with saturated aqueous NaHCO₃ solution (3×) and water, dried over Na₂SO₄, filtered and concentrated in vacuo. The residue is purified by flash chromatography on silica gel. Elution with hexanes-EtOAc (70:30) affords Int 2 as a clear oil (62% yield): ¹H NMR (CDCl₃) δ 7.4-7.2, 7.0, 6.0, 4.2, 3.8, 3.4, 2.1-1.8, 1.3.

Step C. Preparation of trans-4-nitro-1-(phenylmethyl)-3-pyrrolidineacetic acid ethyl ester (Int 3).

A solution of Int 1 (6.81 g, 34.9 mmol) and Int 2 (7.65 g, 34.9 mmol) in EtOH (70 mL) stirs at rt for 15 h and is then concentrated in vacuo. The residue is diluted with ether (100 mL) and saturated aqueous NaHCO₃ solution (100 mL). The organic layer is separated and dried over Na₂SO₄, filtered and concentrated in vacuo. The crude product is purified by flash chromatography on silica gel. Elution with hexanes-EtOAc (85:15) affords Int 3 as a clear oil (76% yield): ¹H NMR (CDCl₃) δ 7.4-7.3, 4.8-4.7, 4.1, 3.8-3.6, 3.3-3.0, 2.7-2.6, 2.4-2.3, 1.2.

Step D. Preparation of trans-4-amino-1-(phenylmethyl)-3-pyrrolidineacetic acid ethyl ester (Int 4).

A mixture of Int 3 (3.28 g, 11.2 mm ol) and RaNi (1.5 g) in EtOH (100 mL) is placed in a Parr bottle and hydrogenated for 4 h under an atmosphere of hydrogen (46 psi) at rt. The mixture is filtered through a pad of Celite, and the solvent is removed in vacuo to afford Int 4 as a clear oil (100% yield): ¹H NMR (300 MHz, CDCl₃) δ 7.3-7.2, 4.1, 3.6, 3.2, 3.0-2.9, 2.8, 2.8-2.6, 2.6-2.4, 2.30-2.2, 1.2.

Step E. Preparation of trans-4-(1,1-dimethylethoxycarbonylamido)-1-(phenylmethyl)-3-pyrrolidineacetic acid ethyl ester (Int 5).

Di-tert-butyldicarbonate (3.67 g, 16.8 mmol) is added to a stirred solution of Int 4 (2.94 g, 11.2 mmol) in CH₂Cl₂ (30 mL) cooled in an ice bath. The reaction is allowed to warm to rt and stirred overnight. The mixture is concentrated in vacuo. The crude product is purified by flash chromatography on silica gel. Elution with hexanes-EtOAc (80:20) affords Int 5 as a white solid (77% yield): ¹H NMR (300 MHz, CDCl₃) δ 7.4-7.2, 5.1-4.9, 4.1, 4.0-3.8, 3.6, 3.2-3.0, 2.8-2.6, 2.5-2.4, 2.3-2.1, 1.4, 1.3.

Step F. Preparation of trans (tert-butoxycarbonylamino)-4-(2-hydroxyethyl)-1-(N-phenylmethyl) pyrrolidine (Int 6).

LiAlH₄ powder (627 mg, 16.5 mmol) is added in small portions to a stirred solution of Int 5 (3.0 g, 8.3 mmol) in anhydrous THF (125 mL) in a −5° C. bath. The mixture is stirred for 20 min in a −5° C. bath, then quenched by the sequential addition of water (0.6 mL), 15% (w/v) aqueous NaOH (0.6 mL) and water (1.8 mL). Excess anhydrous K₂CO₃ is added, and the mixture is stirred for 1 h, then filtered. The filtrate is concentrated in vacuo. The residue is purified by flash chromatography on silica gel. Elution with EtOAc affords Int 6 as a white solid (94% yield): ¹H NMR (CDCl₃) δ 7.4-7.3, 5.3-5.2, 4.1-4.0, 3.9-3.7, 3.3-3.2, 2.8-2.7, 2.3-2.1, 1.7, 1.5.

Int 6 is a racemic mixture that can be resolved via chromatography using a Diacel chiral pack AD column. From the two enantiomers thus obtained, the (+)-enantiomer, [α]²⁵_{D}+35 (c 1.0, MeOH), gives rise to the corresponding enantiomerically pure exo-4-S final compounds, whereas the (−)-enantiomer, [α]²⁵_{D}-4 (c 0.98, MeOH), gives rise to enantiomerically pure exo-4-R final compounds. The methods described herein use the (+)-enantiomer of Int 6 to obtain the enantiomerically pure exo-4-S final compounds. However, the methods used are equally applicable to the (−)-enantiomer of Int 6, making non-critical changes to the methods provided herein to obtain the enantiomerically pure exo-4-R final compounds.

Step G. Preparation of exo 3-(tert-butoxycarbonylamino)-1-azabicyclo[2.2.1]heptane (Int 7).

TEA (8.0 g, 78.9 mml) is added to a stirred solution of Int 6 (2.5 g, 7.8 mmol) in CH₂Cl₂ (50 mL), and the reaction is cooled in an ice-water bath. CH₃SO₂Cl (5.5 g, 47.8 mmol) is then added dropwise, and the mixture is stirred for 10 min in an ice-water bath. The resulting yellow mixture is diluted with saturated aqueous NaHCO₃ solution, extracted with CH₂Cl₂ several times until no product remains in the aqueous layer by TLC. The organic layers are combined, washed with brine, dried over Na₂SO₄ and concentrated in vacuo. The residue is dissolved in EtOH (85 mL) and is heated to reflux for 16 h. The reaction mixture is allowed to cool to rt, transferred to a Parr bottle and treated with 10% Pd/C catalyst (1.25 g). The bottle is placed under an atmosphere of hydrogen (53 psi) for 16 h. The mixture is filtered through Celite, and fresh catalyst (10% Pd/C, 1.25 g) is added. Hydrogenolysis continues overnight. The process is repeated three more times until the hydrogenolysis is complete. The final mixture is filtered through Celite and concentrated in vacuo. The residue is purified by flash chromatography on silica gel. Elution with CHCl₃-MeOH—NH₄OH (90:9.5:0.5) affords Int 7 as a white solid (46% yield): ¹H NMR (CDCl₃) δ 5.6-5.5, 3.8-3.7, 3.3-3.2, 2.8-2.7, 2.0-1.8, 1.7-1.5, 1.5.

Step H. Preparation of exo-3-amino-1-azabicyclo[2.2.1]heptane bis(hydro-para-toluenesulfonate).

Para-toluenesulfonic acid monohydrate (1.46 g, 7.68 mmol) is added to a stirred solution of Int 7 (770 mg, 3.63 mmol) in EtOH (50 mL). The reaction mixture is heated to reflux for 10 h, followed by cooling to rt. The precipitate is collected by vacuum filtration and washed with cold EtOH to give exo-[2.2.1]-Amine as a white solid (84% yield): ¹H NMR (CD₃OD) δ 7.7, 7.3, 3.9-3.7, 3.7-3.3, 3.2, 2.4, 2.3-2.2, 1.9-1.8. The corresponding amines can be obtained by using the resolved Int 6 to give 10 exo-(4R)-[2.2.1]-3-Amine and exo-(4S)-[2.2.1]-3-Amine.

Synthesis of endo-3-amino-1-azabicyclo[2.2.1]heptane as the bis(hydro para-toluenesulfonate) salt:

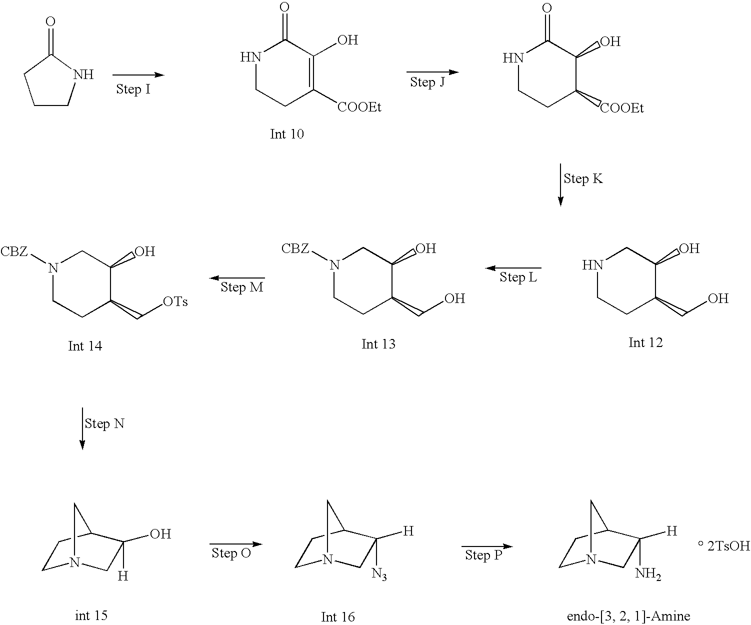

Step I. Preparation of ethyl 5-hydroxy-6-oxo-1,2,3,6-tetrahydropyridine-4-carboxylate (Int 10).

Absolute EtOH (92.0 mL, 1.58 mol) is added to a mechanically stirred suspension of potassium ethoxide (33.2 g, 395 mmol) in dry toluene (0.470 L). When the mixture is homogeneous, 2-pyrrolidinone (33.6 g, 395 mmol) is added, and then a solution of diethyl oxalate (53.1 mL, 390 mmol) in toluene (98 mL) is added via an addition funnel. After complete addition, toluene (118 mL) and EtOH (78 mL) is added sequentially. The mixture is heated to reflux for 18 h. The mixture is cooled to rt and aqueous HCl (150 mL of a 6.0 M solution) is added. The mixture is mechanically stirred for 15 min. The aqueous layer is extracted with CH₂Cl₂, and the combined organic layers are dried over MgSO₄, filtered and concentrated in vacuo to a yellow residue. The residue is recrystallized from EtOAc to afford Int 10 as a yellow solid (38% yield): ¹H NMR (CDCl₃) δ 11.4, 7.4, 4.3, 3.4, 2.6, 1.3.

Step J. Preparation of ethyl cis-3-hydroxy-2-oxopiperidine-4-carboxylate (Int 11).

A mixture of Int 10 (15 g, 81 mmol) and 5% rhodium on carbon (2.0 g) in glacial acetic acid is placed under an atmosphere of hydrogen (52 psi). The mixture is shaken for 72 h. The mixture is filtered through Celite, and the filtrate is concentrated in vacuo to afford Int 11 as a white solid (98% yield): ¹H NMR (CDCl₃) δ 6.3, 4.2, 4.0-3.8, 3.4, 3.3-3.2, 2.2, 1.3.

Step K. Preparation of cis-4-(hydroxymethyl)piperidin-3-ol (Int 12).

Int 11 (3.7 g, 19.9 mmol) as a solid is added in small portions to a stirred solution of LiAlH₄ in THF (80 mL of a 1.0 M solution) in an ice-water bath. The mixture is warmed to rt, and then the reaction is heated to reflux for 48 h. The mixture is cooled in an ice-water bath before water (3.0 mL, 170 mmol) is added dropwise, followed by the sequential addition of NaOH (3.0 mL of a 15% (w/v) solution) and water (9.0 mL, 500 mmol). Excess K₂CO₃ is added, and the mixture is stirred vigorously for 15 min. The mixture is filtered, and the filtrate is concentrated in vacuo to afford Int 12 as a yellow powder (70% yield): ¹H NMR (DMSO-d₆) δ 4.3, 4.1, 3.7, 3.5-3.2, 2.9-2.7, 2.5-2.3, 1.5, 1.3.

Step L. Preparation of benzyl cis-3-hydroxy-4-(hydroxymethyl)piperidine-1-carboxylate (Int 13).

N-(benzyloxy carbonyloxy)succinimide (3.04 g, 12.2 mmol) is added to a stirred solution of Int 12 (1.6 g, 12.2 mmol) in saturated aqueous NaHCO₃ (15 mL) at rt. The mixture is stirred at rt for 18 h. The organic and aqueous layers are separated. The aqueous layer is extracted with ether (3×). The combined organic layers are dried over anhydrous K₂CO₃, filtered and concentrated in vacuo to afford Int 13 as a yellow oil (99% yield): ¹H NMR (CDCl₃) δ 7.4-7.3, 5.2, 4.3, 4.1, 3.8-3.7, 3.0-2.8, 2.1, 1.9-1.7, 1.4.

Step M. Preparation of benzyl cis-3-hydroxy-4-[(4-methylphenyl)sulfonyl oxymethyl]piperidine-1-carboxylate (Int 14).

Para-toluenesulfonyl chloride (1.0 g, 5.3 mmol) is added to a stirred solution of Int 13 (3.6 g, 5.3 mmol) in pyridine (10 mL) in a −15° C. bath. The mixture is stirred for 4 h, followed by addition of HCl (4.5 mL of a 6.0 M solution). CH₂Cl₂ (5 mL) is added. The organic and aqueous layers are separated. The aqueous layer is extracted with CH₂Cl₂. The combined organic layers are washed with brine, dried over MgSO₄, filtered and concentrated in vacuo to afford Int 14 as a colorless oil (78% yield): ¹H NMR (CDCl₃) δ 7.8, 7.4-7.2, 5.1, 4.3-4.2, 4.1, 3.9-3.8, 2.9-2.7, 2.4, 1.9, 1.6-1.3.

Step N. Preparation of exo-1-azabicyclo[2.2.1]heptan-3-ol (Int 15).

A mixture of Int 14 (3.6 g, 8.6 mmol) and 10% Pd/C catalyst (500 mg) in EtOH (50 mL) is placed under an atmosphere of hydrogen. The mixture is shaken for 16 h. The mixture is filtered through Celite. Solid NaHCO₃ (1.1 g, 13 mmol) is added to the filtrate, and the mixture is heated in an oil bath at 50° C. for 5 h. The solvent is removed in vacuo. The residue is dissolved in saturated aqueous K₂CO₃ solution. Continuous extraction of the aqueous layer using a liquid-liquid extraction apparatus (18 h), followed by drying the organic layer over anhydrous K₂CO₃ and removal of the solvent in vacuo affords Int 15 as a white solid (91% yield): ¹H NMR δ3.8, 3.0-2.8, 2.6-2.5, 2.4-2.3, 1.7, 1.1.

Step O. Preparation of endo-3-azido-1-azabicyclo[2.2.1]heptane (Int 16).

To a mixture of Int 15 (1.0 g, 8.9 mmol) and triphenyl phosphine (3.0 g, 11.5 mmol) in toluene-THF (50 mL, 3:2) in an ice-water bath are added sequentially a solution of hydrazoic acid in toluene (15 mL of ca. 2 M solution) and a solution of diethyl azadicarboxylate (1.8 mL, 11.5 mmol) in toluene (20 mL). The mixture is allowed to warm to rt and stir for 18 h. The mixture is extracted with aqueous 1.0M HCl solution. The aqueous layer is extracted with EtOAc, and the combined organic layers are discarded. The pH of the aqueous layer is adjusted to 9 with 50% aqueous NaOH solution. The aqueous layer is extracted with CH₂Cl₂ (3×), and the combined organic layers are washed with brine, dried over Na₂SO₄, filtered and concentrated in vacuo. The crude product is purified by flash chromatography on silica gel. Elution with CHCl₃-MeOH—NH₄OH (92:7:1) affords Int 16 as a colorless oil (41% yield): ¹H NMR (CDCl₃) δ 4.1, 3.2, 2.8, 2.7-2.5, 2.2, 1.9, 1.5.

Step P. Preparation of endo-3-amino-1-azabicyclo[2.2.1]heptane bis(hydro-para-toluenesulfonate).

A mixture of Int 16 (250 mg, 1.8 mmol) and 10% Pd/C catalyst (12 mg) in EtOH (10 mL) is placed under an atmosphere of hydrogen (15 psi). The mixture is stirred for 1 h at rt. The mixture is filtered through Celite, and the filtrate is concentrated in vacuo. The residue is dissolved in EtOH (10 mL) and para-toluenesulfonic acid monohydrate (690 mg, 3.7 mmol) is added. The mixture is stirred for 30 min, and the precipitate is filtered. The precipitate is washed sequentially with cold EtOH and ether. The precipitate is dried in vacuo to afford endo-[2.2.1]-Amine as a white solid (85% yield): ¹H NMR (CD₃OD) δ 7.7, 7.3, 4.2, 3.9, 3.6-3.4, 3.3-3.2, 2.4, 2.3, 2.1.

Preparation of the 3.2.1-Amine:

exo-1-Azabicyclo[3.2.1]octan-3-amine dihydrochloride (exo-[3.2.1]-Amine):

A mixture of 1-azabicyclo[3.2.1]octan-3-one hydrochloride (2.80 g, 17.3 mmol), ethanol (25 mL), and hydroxylamine hydrochloride (1.56 g, 22.4 mmol) is treated with sodium acetate trihydrate (7.07 g, 51.2 mmol). The mixture is stirred for 3 h and evaporated in vacuo. The residue is diluted with CH₂Cl₂, treated with charcoal, filtered and evaporated. The resulting material is taken up in 1-propanol (45 mL) and heated in a 100° C. oil bath. The solution is treated with sodium metal (6.4 g in portions). Heating is continued for 3 h and the mixture cooled to rt. Water is added carefully and the organic layer is extracted, dried (MgSO₄), filtered, acidified with MeOH/HCl(g), and evaporated. 2-Propanol is added and the resulting solid is filtered and dried in vacuo to give exo-[3.2.1]-Amine in 49% yield. MS for C₇H₁₄N₂.(HCl)₂ (ESI) (M+H)⁺m/z=127.

endo-1-Azabicyclo[3.2.1]octan-3-amine dihydrochloride (endo-[3.2.1]-Amine):

A mixture of 1-azabicyclo[3.2.1]octan-3-one hydrochloride (2.80 g, 17.3 mmol), ethanol (25 mL), and hydroxylamine hydrochloride (1.56 g, 22.4 mmol) is treated with sodium acetate trihydrate (7.07 g, 51.2 mmol). The mixture is stirred for 3 h and evaporated in vacuo. The residue is diluted with CH₂Cl₂, treated with charcoal, filtered and evaporated. The resulting oxime (3.1 mmol) is treated with acetic acid (30 mL) and hydrogenated at 50 psi over PtO₂ (50 mg) for 12 h. The mixture is then filtered and evaporated. The residue is taken up in a minimal amount of water (6 mL) and the pH is adjusted to >12 using solid NaOH. The mixture is then extracted with ethyl acetate (4×25 n-dL), dried over MgSO₄, filtered, treated with ethereal HCl, and evaporated to give endo-[3.2.1]-Amine.

1-Azabicyclo[3.2.1]octan-3-amine:

Preparation of the 3R,5R-[3.2.1]-Amine:

This amine can also be prepared according to the following method:

(3S)-1-[(S)-1-Phenethyl]-5-oxo-3-pyrrolidine-carboxylic Acid:

According to the literature procedure (Nielsen et al. J. Med. Chem 1990, 70-77), a mixture of itaconic acid (123.17 g, 946.7 mmol) and (S)-(−)-α-methyl benzylamine (122.0 mL, 946.4 mmol) are heated (neat) in a 160° C. oil bath for 4 h. Upon cooling, MeOH (˜200 mL) is added and the resulting solid collected by filtration. The solid is treated with EtOH (˜700 mL) and warmed using a steam bath until ˜450 m-tL solvent remained. After cooling to rt, the solid is collected and dried to afford 83.2 g as a crystalline solid: [α]²⁵_{D}=−80 (c 0.97, DMSO). ¹H NMR (400 MHz, DMSO-d₆) δ 12.66, 7.20-7.40, 5.23, 3.40-3.55, 3.10-3.25, 2.40-2.65, 1.45; MS (EI) m/z 233 (M⁺).

(3S)-1-[(S)-1-Phenethyl]-3-(hydroxymethyl)pyrrolidine:

A suspension (3S)-1-[(S)-1-phenethyl]-5-oxo-3-pyrrolidine-carboxylic acid (82.30 g, 352.8 mmol) in Et₂O (200 nL) is added in small portions to a slurry of LiAlH₄ (17.41 g, 458.6 mmol) in Et₂O (700 mL). The mixture begins to reflux during the addition. The addition funnel containing the suspension is rinsed with Et₂O (2×50 mL), and the mixture is heated in a 50° C. oil bath for an additional 2 h and first allowed to cool to rt and then further cooled using an ice bath. The mixture is carefully treated with H₂O (62 mL). The resulting precipitate is filtered, rinsed with Et₂O, and discarded. The filtrate is concentrated to a yellow oil. When EtOAc is added to the oil, a solid began to form. Hexane is then added, and the mixture is filtered and the solid is dried to afford 43.3 g. [α]²⁵_{D}=−71 (c 0.94, CHCl₃); ¹H NMR (400 MHz, CDCl₃) δ 7.20-7.45, 3.60-3.70, 3.40-3.60, 3.19, 3.05-3.15, 2.35-2.55, 2.25-2.35, 1.95-2.10, 1.75-1.90, 1.42; HRMS (FAB) calcd for C₁₃H₁₉NO (MH⁺) 206.1545, found 206.1532.

(3R)-1-[(S)-1-Phenethyl]-3-(cyanomethyl)pyrrolidine:

A solution of (3S)-1-[(S)-1-phenethyl]-3-(hydroxymethyl)pyrrolidine (42.75 g, 208.23 mmol) in chloroform (350 ml) is heated to reflux under N₂. The solution is treated with a solution of thionyl chloride (41.8 mL, 573 mmol) in chloroform (40 mL) dropwise over 45 min. The mixture is stirred for an additional 30 min, is cooled and concentrated. The residue is diluted with H₂O (˜200 mL), 1 N NaOH is added until a pH ˜8 (pH paper). A small portion (˜50 mL) of sat. NaHCO₃ is added and the basic mixture is extracted with EtOAc (3×400 mL), washed with brine, dried over MgSO₄, filtered and concentrated to give 46.51 g of (3S)-1-[(S)-1-phenethyl]-3-(chloromethyl)pyrrolidine: MS (ESI+) m/z 224.2 (MH⁺). The chloride (46.35 g, 208.0 mmol) is transferred to a flask, DMSO (200 mL) is added, and the solution is treated with NaCN (17.84 g, 363.9 mmol). The mixture is heated under N₂ in a 100° C. oil bath overnight and is cooled. The brown mixture is poured into H₂O (300 mL) and is extracted with EtOAc (1000 mL in portions). The combined organic layer is washed with H₂O (6×˜50 mL), brine (˜100 mL), dried (MgSO₄), filtered and concentrated to give 40.61 g of an oil: ¹H NMR (400 MHz, CDCl₃) δ 7.20-7.40, 3.26, 2.70-2.85, 2.40-2.60, 2.27, 2.10-2.20, 1.50-1.70, 1.41; MS (ESI+) for m/z 215.2 (M+H⁺).

(3R)-Methyl 1-[(S)-1-phenylethyl]pyrrolidine-3-acetate:

Acetyl chloride (270 mL, 3.8 mol) is carefully added to a flask containing chilled (0° C.) methanol (1100 nL). After the addition is complete, the acidic solution is stirred for 45 min (0° C.) and then (3R)-1-[(S)-1-phenethyl]-3-(cyanomethyl)pyrrolidine (40.50 g, 189.0 mmol) in methanol (200 mL) is added. The ice bath is removed and the mixture is stirred for 100 h at rt. The resulting suspension is concentrated. Water (˜600 mL) is added, the mixture stirred for 45 min and then the pH is adjusted (made basic) through the addition of ˜700 mL sat. aq. NaHCO₃. The mixture is extracted with EtOAc (3×300 mL). The combined organic layers are washed with brine, dried (MgSO₄), filtered through celite and concentrated to give 36.86 g as an oil: ¹H NMR (400 MHz, CDCl₃) δ 7.20-7.40, 3.69, 3.30-3.40, 2.85-2.95, 2.40-2.70, 2.00-2.20, 1.10-1.65; MS (ESI+) m/z 248.2 (M+H⁺).

(5R)-1-Azabicyclo[3.2.1]octan-3-one hydrochloride:

A solution of (3R)-methyl 1-[(S)-1-phenylethyl]pyrrolidine-3-acetate (25.72 g, 104.0 mmol) in THF (265 m]L) is cooled under N₂ in a CO₂/acetone bath. Next, ICH₂Cl (22.7 mL, 312.0 mm-ol) is added, and the mixture stirred for 30 min. A solution of 2.0M lithium diisopropylamide (heptane/THF/ethylbenzene, 156 mL, 312 mmol) is added slowly over 30 min. The internal temperature reached a maximum of −40° C. during this addition. After 1 h, sat. NH₄Cl (100 mL) is added and the mixture is allowed to warm to rt. The organic layer is separated, dried (MgSO₄), filtered and concentrated. The resulting foam is chromatographed (300 g SiO2, CHCl₃-MeOH—NH₄OH (89:10:1) followed by CHCl₃-MeOH (3:1). The product fractions are pooled and concentrated to afford (5R)-3-oxo-1-[(1S)-1-phenylethyl]-1-azoniabicyclo[3.2.1]octane chloride (10.12 g) as a foam (MS (ESI+) ni/z 230.1 (M+H⁺). This foam (10.1 g, 38 mmol) is taken up in MeOH (500 mL), 10% Pd(C) (3.0 g) added and the mixture is hydrogenated (45 psi) overnight. The mixture is filtered and re-subjected to the reduction conditions (9.1 g, 10% Pd/C, 50 psi). After 5 h, TLC indicates the consumption of the (5R)-3-oxo-1-[(1S)-1-phenylethyl]-1-azoniabicyclo[3.2.1]octane chloride. The mixture is filtered, concentrated and triturated (minimal iPrOH) to give 3.73 g in two crops, as a solid: [a]25 D=33 (c 0.97, DMSO); HRMS (FAB) calcd for C₇H₁₁NO (M+H⁺) 126.0919, found 126.0937.

(3R,5R)-1-azabicyclo[3.2.1]octan-3-amine dihydrochloride:

To a flask containing (5R)-1-azabicyclo[3.2.1]octan-3-one hydrochloride (3.64 g, 22.6 mmol), hydroxylamine hydrochloride (2.04 g, 29.4 mmol), and ethanol (130 mL) is added sodium acetate trihydrate (9.23 g, 67.8 mmol). The mixture stirred for 3 h and is filtered and concentrated. The resulting white solid is taken up in n-propanol (100 mL) and sodium (˜13.6 g, 618 mmol) is added in 20-25 portions. The reaction spontaneously begins to reflux, and the reaction is heated in an oil bath (100° C.). The addition is complete in ˜20 min and the mixture solidifies after ˜40 min. The oil bath is removed and n-propanol (2×25 mL) is added dissolving the remaining sodium metal. The mixture is carefully quenched through the dropwise addition of H₂O (100 mL). Saturated aq. NaCl (20 mL) is added, and the layers are separated. The organic layer is dried (MgSO₄), filtered, treated with freshly prepared MeOH/HCl, and concentrated. The resulting solid is triturated with 30 nL EtOH, filtered and dried in vaccuo to afford 3.51 g as a white solid: [α]²⁵_{D}=−3 (c 0.94, DMSO); ¹H NMR (400 MHz, DMSO-d₆) δ 3.60-3.80, 2.95-3.10, 2.65-2.75, 1.90-2.15, 1.70-1.90; HRMS (FAB) calcd for C₇H₁₄N₂ (M+H⁺) 127.1235, found 127.1235.

Preparation of tert-butyl (1S, 2R, 4R)-2-amino-7-azabicyclo[2.2.1]heptane-7-carboxylate:

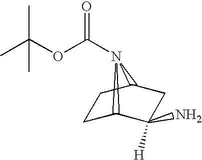

Preparation of methyl-3-bromo-propiolate:

Methyl propiolate (52 ml, 0.583 mol) is combined with recrystallized N-bromo-succinimide (120 g, 0.674 mol) in 1,700 ml acetone under nitrogen. The solution is treated with silver nitrate (9.9 g, 0.0583 mol) neat in a single lot and the reaction is stirred 6 h at RT. The acetone is removed under reduced pressure (25° C., bath temperature) to provide a gray slurry. The slurry is washed with 2×200 ml hexane, the gray solid is removed by filtration, and the filtrate is concentrated in vacuo to provide 95 g of a pale yellow oily residue. The crude material is distilled via short path under reduced pressure (65° C., about 25 mm Hg) into a dry ice/acetone cooled receiver to give 83.7 g (88%) of methyl-3-bromo-propiolate as a pale yellow oil. Anal. calc'd for C₄H₃BrO₂: C, 29.48; H, 1.86. Found: C, 29.09; H, 1.97.

Preparation of 7-tert-butyl 2-methyl 3-bromo-7-azabicyclo[2.2.1]hepta-2,5-diene-2,7-dicarboxylate.

Methyl-3-bromo-propiolate (83.7 g, 0.513 mol) is added to N-t-butyloxy-pyrrole (430 ml, 2.57 mol) under nitrogen. The dark mixture is warmed in a 90° C. bath for 30 h, is cooled, and the bulk of the excess N-t-butyloxy-pyrrole is removed in vacuo using a dry ice/acetone condenser. The dark oily residue is chromatographed over 1 kg silica gel (230-400 mesh) eluting with 0-15% EtOAc/hexane. The appropriate fractions are combined and concentrated to afford 97 g (57%) of 7-tert-butyl 2-methyl 3-bromo-7-azabicyclo[2.2.1]hepta-2,5-diene-2,7-dicarboxylate as a dark yellow oil. HRMS (FAB) calc'd for C₁₃H₁₆BrNO₄+H: 330.0341, found 330.0335 (M+H)⁺.

Preparation of (+/−) endo-7-tert-butyl 2-methyl 7-azabicyclo[2.2.1]heptane-2,7-dicarboxylate.

7-tert-Butyl 2-methyl 3-bromo-7-azabicyclo[2.2.1]hepta-2,5-diene-2,7-dicarboxylate (97 g, 0.294 mol) is added to 10% Pd/C (6.8 g) in 900 ml absolute EtOH in a PARR bottle. The suspension is diluted with a solution of NaHCO₃ (25 g, 0.301 mol) in 250 ml water and the mixture is hydrogenated at 50 PSI for 2.5 h. The catalyst is removed by filtration, is washed with fresh EtOH, and the filtrate is concentrated in vacuo to give a residue. The residue is partitioned between 1×200 ml saturated NaHCO₃ and CH₂Cl₂ (4×100 ml). The combined organic layer is dried over 1:1 anhydrous K₂CO₃/anhydrous MgSO₄ and concentrated in vacuo to afford 72.8 g (98%) of (+/−) endo-7-tert-butyl 2-methyl 7-azabicyclo[2.2.1]heptane-2,7-dicarboxylate. MS (EI) for C₁₄H₂₂O₄, m/z: 255 (M)⁺.

Preparation of (+/−) exo-7-(tert-butoxycarbonyl)-7-azabicyclo[2.2.1]heptane-2-carboxylic acid.

(+/−)Endo-7-tert-butyl 2-methyl 7-azabicyclo[2.2.1]heptane-2,7-dicarboxylate (72.8 g, 0.285 mol) is dissolved in 1000 ml dry MeOH in a dried flask under nitrogen. The solution is treated with solid NaOMe (38.5 g, 0.713 mol) neat, in a single lot and the reaction is warmed to reflux for 4 h. The mixture is cooled to 0° C., is treated with 400 ml water, and the reaction is stirred Ih as it warms to RT. The mixture is concentrated in vacuo to about 400 ml and the pH of the aqueous residue is adjusted to 4.5 with 12N HCl. The precipitate is collected and dried. The tan, slightly tacky solid is washed with 2×100 ml 60% ether in hexane and is dried to provide 47 g (68%) of (+/−) exo-7-(tert-butoxycarbonyl)-7-azabicyclo[2.2.1]heptane-2-carboxylic acid as an off-white powder. HRMS (FAB) calc'd for C₁₂H₁₉NO₄+H: 242.1392, found 242.1390 (M+H)⁺.

Preparation of (+/−) exo-tert-butyl 2-{[(benzyloxy)carbonyl]amino}-7-azabicyclo[2.2.1]heptane-7-carboxylate.

(+/−)Exo-7-(tert-butoxycarbonyl)-7-azabicyclo[2.2. I]heptane-2-carboxylic acid (103.9 g, 0.430 mol) is combined with TEA (60 ml, 0.430 mol) in 1200 ml dry toluene in a dry flask under nitrogen. The solution is treated drop-wise with diphenylphosphoryl azide (92.8 ml, 0.430 mol), and is allowed to stir for 20 min at RT. The mixture is treated with benzyl alcohol (47.9 ml, 0.463 mol), and the reaction is stirred overnight at 55° C. The mixture is cooled, is extracted successively with 2×500 ml 5% citric acid, 2×500 ml water, 2×500 ml saturated sodium bicarbonate, and 500 ml saturated NaCl. The organic layer is dried over anhydrous MgSO₄ and concentrated in vacuo to an amber oil. The crude material is chromatographed over 900 g silica gel (230-400 mesh), eluting with 10-30% EtOAc/hexane. The appropriate fractions are combined and concentrated to give 106 g (71%) of (+/−) exo-tert-butyl 2-{[(benzyloxy)carbonyl]amino}-7-azabicyclo[2.2.1]heptane-7-carboxylate as a pale oil.

¹H NMR (CDCl₃) δ 1.29-1.60, 1.44, 1.62-2.01, 3.76-3.88, 4.10, 4.24, 5.10, 7.36 ppm.

Preparation of (+/−) exo-tert-butyl 2-amino-7-azabicyclo[2.2.1]heptane-7-carboxylate.

(+/−) Exo-tert-Butyl 2-{[(benzyloxy)carbonyl]amino}-7-azabicyclo[2.2.1]heptane-7-carboxylate (1.5 g, 4.33 mmol) is combined with 10% Pd/C (150 mg) in 40 ml EtOH in a 250 ml Parr shaker bottle. The mixture is hydrogenated at 50 PSI for 1.5 h. The catalyst is removed by filtration and the filtrate is concentrated in vacuo. The crude material is chromatographed over 30 g silica gel (230-400 mesh), eluting with 7% MeOH/CH₂Cl₂+1% conc. NH₄OH. The appropriate fractions are combined and concentrated to provide 606 mg (66%) of (+/−) exo-tert-butyl 2-amino-7-azabicyclo[2.2.1]heptane-7-carboxylate. HRMS (FAB) calcd for C₁₁H₂₀N₂O₂+H: 213.1603, found 213.1580 (M+H)⁺. This racemic mixture will be referenced as (+/−)-7-aza-[2.2.1]-Amine.

Resolution of racemic carboxylate mixture:

The isolated (+/−) exo-tert-butyl 2-{[(benzyloxy)carbonyl]amino}-7-azabicyclo[2.2.1]heptane-7-carboxylate is resolved via preparative chiral HPLC (50×500 mm Chiralcel OJ column, 30 deg. C, 70 mL/min. 10/90 (v/v) isopropanol/heptane). The resolution affords 40 g of tert-butyl (1S, 2R, 4R)-(+)-2{[(benzyloxy)carbonyl]amino}-7-azabicyclo[2.2.1]heptane-7-carboxylate and 42 g of tert-butyl-(1R, 2S, 4S)(−)-2 {[(benzyloxy)carbonyl]amino}-7-azabicyclo[2.2.1]heptane-7-carboxylate.

The 2R enantiomer is triturated with 40 ml ether followed by 40 ml hexane (to remove lingering diastereo and enantiomeric impurities) and is dried to afford 30 g (56%) of purified tert-butyl (1S, 2R, 4R)-(+)-2{[(benzyloxy)carbonyl]amino}-7-azabicyclo[2.2.1]heptane-7-carboxylate with 99% enantiomeric excess. MS (EI) for C₁₉H₂₆N₂O₄, m/z: 346 (M)⁺. [α]²⁵ _{D}=22, (c 0.42, chloroform).

The 2S enantiomer is triturated with 40 ml ether followed by 40 ml hexane to give 35 g (66%) of purified tert-butyl (1R, 2S, 4S)-(−)-2 {[(benzyloxy)carbonyl]amino}-7-azabicyclo[2.2.1]heptane-7-carboxylate with 99% enantiomeric excess. MS (EI) for C₁₉H₂₆N₂O₄, m/z: 346 (M)⁺. [α]²⁵_{D}=−23, (c 0.39, chloroform).

Preparation of tert-butyl-(1S, 2R, 4R)-(+)-2-amino-7-azabicyclo[2.2.1]heptane-7-carboxylate ((2R)-7-aza-[2.2.1]-Amine).

tert-Butyl (1S, 2R, 4R)-(+)-2{[(benzyloxy)carbonyl]amino}-7-azabicyclo[2.2.1]heptane-7-carboxylate (9.5 g, 27.4 mmol) is combined with 950 mg 10% Pd/C in 75 ml absolute EtOH in a 500 ml Parr bottle. The reaction mixture is hydrogenated at 50 PSI for 3 h, the catalyst is removed by filtration, and the filter cake is washed with MeOH. The filtrate is concentrated in vacuo to give 6.4 g of a residue. The crude material is chromatographed over 200 g silica gel (230-400 mesh) eluting with 7% CH₃OH/CHCl₃ containing 1% conc. NH₄OH. The appropriate fractions are combined and concentrated to give 5.61 g (96%) of tert-butyl-(1S, 2R, 4R)-(+)-2-amino-7-azabicyclo[2.2.1]heptane-7-carboxylate as a pale oil. MS (EI) for C₁₁H₂₀N₂O₂, m/z: 212 (M)⁺. [α]²⁵D=9, (c 0.67, CHCl₃). This compound will be referenced as (2R)-7-aza-[2.2.1]-Amine.

### EXAMPLE 1

### N-[(3R)-1-azabicyclo[2.2.2]oct-3-yl]-4-[¹²³I]iodo-1H-pyrazole-1-carboxamide hydrochloride:

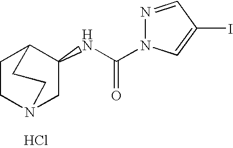

Phenyl chloroformate (0.75 mL, 6.0 mmol) is added dropwise to a solution of 4-iodopyrazole (1.05 g, 5.4 mmol) and triethylamine (0.9 mL, 6.5 mmol) in 15 mL CH₂Cl₂. The reaction is stirred at RT. After 60 h, water is added. The mixture is extracted with CH₂Cl₂, dried (MgSO₄), filtered and concentrated. Hexane is added and the solvent is removed in vacuo. A white solid forms on standing to provide 1.6 g (95%) of phenyl 4-iodo-1H-pyrazole-1-carboxylate. MS (EI) m/z 315.1 (M⁺).

Phenyl 4-iodo-1H-pyrazole-1-carboxylate (1.6 g, 5.2 mmol) and (R)-(+)-3-aminoquinuclidine dihydrochloride (1.0 g, 5.2 mmol) are suspended in 10 mL DMF. DIEA (2.7 mL, 15.5 mmol) is added dropwise. After 36 h, the solvent is removed and the residue is taken up in 1N NaOH and CHCl₃. The aqueous layer is extracted with CHCl₃, dried (MgSO₄), filtered and concentrated. The residue is purified by chromatography (Biotage 40S, 90:9:1 CHCl₃/MeOH/NH₄OH) to provide 1.66 g (93%) of the product as a white solid. A portion of the material is converted into the hydrochloride salt and recrystallized from MeOH/EtOAc. HRMS (FAB) calcd for C₁₁H₁₅IN₄O+H 347.0370, found 347.0357.

N-[(3R)-1-azabicyclo[2.2.2]oct-3-yl]-4-iodo-1H-pyrazole-1-carboxamide can then be converted to N-[(3R)-1-azabicyclo[2.2.2]oct-3-yl]-4-[¹²³I]iodo-1H-pyrazole-1-carboxamide using procedures discussed herein.

Materials and Methods for Identifying Binding Constants Using Non-Labeled Agonsits

Membrane Preparation. Male Sprague-Dawley rats (300-350 g) are sacrificed by decapitation and the brains (whole brain minus cerebellum) are dissected quickly, weighed and homogenized in 9 volumes/g wet weight of ice-cold 0.32 M sucrose using a rotating pestle on setting 50 (10 up and down strokes). The homogenate is centrifuged at 1,000×g for 10 minutes at 4° C. The supernatant is collected and centrifuged at 20,000×g for 20 minutes at 4° C. The resulting pellet is resuspended to a protein concentration of 1-8 mg/nL. Aliquots of 5 mL homogenate are frozen at −80° C. until needed for the assay. On the day of the assay, aliquots are thawed at room temperature and diluted with Kreb's −20 mM Hepes buffer pH 7.0 (at room temperature) containing 4.16 mM NaHCO₃, 0.44 mM KH₂PO₄, 127 mM NaCl, 5.36 mM KCl, 1.26 mM CaCl₂, and 0.98 mM MgCl₂, so that 25-150 μg protein are added per test tube. Proteins are determined by the Bradford method (Bradford, M. M., Anal. Biochem., 72, 248-254, 1976) using bovine serum albumin as the standard.

Binding Assay. For saturation studies, 0.4 mL homogenate are added to test tubes containing buffer and various concentrations of radioligand ([³H]-MLA), and are incubated in a final volume of 0.5 mL for 1 hour at 25° C. Nonspecific binding was determined in tissues incubated in parallel in the presence of 0.05 ml MLA for a final concentration of 1 μM MLA, added before the radioligand ([³H]-MLA). In competition studies, agonists are added in increasing concentrations to the test tubes before addition of 0.05 ml [³H]-MLA for a final concentration of 3.0 to 4.0.nM [³H]-MLA. The incubations are terminated by rapid vacuum filtration through Whatman GF/B glass filter paper mounted on a 48 well Brandel cell harvester. Filters are pre-soaked in 50 mM Tris HCl pH 7.0-0.05% polyethylenimine. The filters are rapidly washed two times with 5 mL aliquots of cold 0.9% saline and then counted for radioactivity by liquid scintillation spectrometry.

Data Analysis. In competition binding studies, the inhibition constant (Ki) was calculated from the concentration dependent inhibition of [³H]-MLA binding obtained from non-linear regression fitting program according to the Cheng-Prusoff equation (Cheng, Y. C. and Prussoff, W. H., Biochem. Pharmacol., 22, p. 3099-3108, 1973). Hill coefficients were obtained using non-linear regression (GraphPad Prism sigmoidal dose-response with variable slope).

Blood-Brain Barrier Penetration

Pharmacokinetics of the agonists (non-radiolabeled compounds of formula I) can be evaluated in mice to determine the ability of each compound to penetrate the blood-brain barrier. Each mouse receives a single intravenous administration at 5 mg/kg. Blood samples are collected by serial sacrifice at 5 min (IV only), 0.5, 1, 2, 4, and 8 h after dosing with two mice per collection time. Blood was placed into tubes containing heparin and centrifuged for plasma. Brain samples were also collected at 0.5 and 1 h increments from the same mouse used for blood collection. Plasma and brain samples were analyzed for drug concentrations using a LC-MS/MMS method. Pharmacokinetics (clearance, volume of distribution, and half-life) were evaluated from the plasma concentration-time data (See Gibaldi and Perrier in Pharmacokinetics, Vol I, 2^{nd} ed, New York: Marcel Dekker, 1982). Compounds having a large volume of distribution will have good distribution into the body tissues. Comparison of the drug concentration in brain and plasma (brain/plasma ratio) provides the direct information of brain penetration. Higher numbers refer to higher brain penetration.

1. A compound of Formula I:
Azabicyclo-N(R₁)—C(═O)—W Formula I
wherein Azabicyclo is 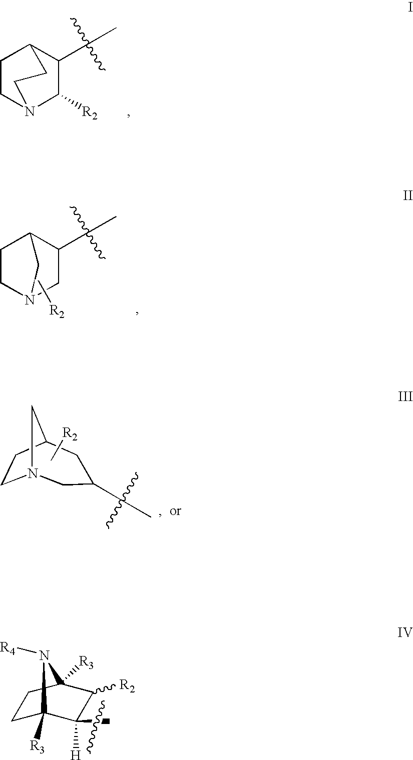
R₁ is H;
R₂ is H or alkyl;
Each R₃ is independently H, alkyl, or substituted alkyl;
R₄ is H, alkyl, an amino protecting group, or an alkyl group having 1-3 substituents selected from F, Cl, Br, 1, —OH, —CN, —NH₂, —NH(alkyl), or —N(alkyl)₂;
R₅ is 5-membered heteroaromatic mono-cyclic moieties containing within the ring 1-3 heteroatoms independently selected from the group consisting of —O—, ═N—, —N(R₁₀)—, and —S—, and having 0-1 substituent selected from R₉ and further having 0-3 substituents independently selected from F, Cl, Br, or I, or R₅ is 9-membered fused-ring moieties having a 6-membered ring fused to a 5-membered ring and having the formula 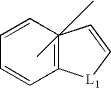
wherein L₁ is O, S, or NR₁₀, 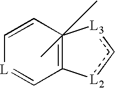
wherein L is CR₁₂ or N, L₂ and L₃ are independently selected from CR₁₂, C(R₁₂)₂, O, S, N, or NR₁₀, provided that both L₂ and L₃ are not simultaneously O, simultaneously S, or simultaneously O and S, or 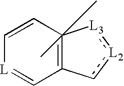
wherein L is CR₁₂ or N, and L₂ and L₃ are independently selected from CR₁₂, O, S, N, or NR₁₀, and each 9-membered fused-ring moiety having 0-1 substituent selected from R₉ and further having 0-3 substituent(s) independently selected from F, Cl, Br, or I, wherein the R₅ moiety attaches to other substituents as defined in formula I at any position as valency allows;
R₆ is 6-membered heteroaromatic mono-cyclic moieties containing within the ring 1-3 heteroatoms selected from ═N— and having 0-1 substituent selected from R₉ and 0-3 substituent(s) independently selected from F, Cl, Br, or I, or R₆ is 10-membered heteroaromatic bi-cyclic moieties containing within one or both rings 1-3 heteroatoms selected from ═N—, including, but not limited to, quinolinyl or isoquinolinyl, each 10-membered fused-ring moiety having 0-1 substituent selected from R₉ and 0-3 substituent(s) independently selected from F, Cl, Br, or I, wherein the R₆ moiety attaches to other substituents as defined in formula I at any position as valency allows;
R₇ is alkyl, substituted alkyl, haloalky, —OR₁₁, —CN, —NO₂, —N(R₈)₂;
Each R₈ is independently H, alkyl, cycloalkyl, heterocycloalkyl, alkyl substituted with 1 substituent selected from R₁₃, cycloalkyl substituted with 1 substituent selected from R₁₃, heterocycloalkyl substituted with 1 substituent selected from R₁₃, haloalkyl, halocycloalkyl, haloheterocycloalkyl, phenyl, or substituted phenyl;
R₉ is alkyl, cycloalkyl, heterocycloalkyl, haloalkyl, halocycloalkyl, haloheterocycloalkyl, —OR₁₄, —SR₁₄, —N(R₁₄)₂, —C(O)R₁₄, —C(O)N(R₁₄)₂, —CN, —NR₁₄C(O)R₁₄, —S(O)₂N(R₁₄)₂, —NR₁₄S(O)₂R₁₄, —NO₂, alkyl substituted with 1-4 substituent(s) independently selected from F, Cl, Br, I, or R₁₃, cycloalkyl substituted with 1-4 substituent(s) independently selected from F, Cl, Br, I, or R₁₃, or heterocycloalkyl substituted with 1-4 substituent(s) independently selected from F, Cl, Br, I, or R₁₃;
R₁₀ is H, alkyl, haloalkyl, substituted alkyl, cycloalkyl, halocycloalkyl, substituted cycloalkyl, phenyl, or phenyl having 1 substituent selected from R₇ and further having 0-3 substituents independently selected from F, Cl, Br, or I;
Each R₁₁ is independently H, alkyl, cycloalkyl, heterocycloalkyl, haloalkyl, halocycloalkyl, or haloheterocycloalkyl;
Each R₁₂ is independently H, F, Cl, Br, I, alkyl, cycloalkyl, heterocycloalkyl, haloalkyl, halocycloalkyl, haloheterocycloalkyl, substituted alkyl, substituted cycloalkyl, substituted heterocycloalkyl, —CN, —NO₂, —OR₁₄, —SR₁₄, —N(R₁₄)₂, —C(O)R₁₄, —C(O)N(R₁₄)₂, —NR₁₄C(O)R₁₄, —S(O)₂N(R₁₄)₂, —NR₁₄S(O)₂RR₁₄, or a bond directly or indirectly attached to the core molecule, provided that there is only one said bond to the core molecule within the 9-membered fused-ring moiety, further provided that where valency allows the fused-ring moiety has 0-1 substituent selected from alkyl, cycloalkyl, heterocycloalkyl, haloalkyl, halocycloalkyl, haloheterocycloalkyl, substituted alkyl, substituted cycloalkyl, substituted heterocycloalkyl, —OR₁₄, —SR₁₄, —N(R₁₄)₂, —C(O)R₁₄, —NO₂, —C(O)N(R₁₄)₂, —CN, —NR₁₄C(O)R₁₄, —S(O)₂N(R₁₄)₂, or —NR₁₄S(O)₂R₁₄, and further provided that the fused-ring moiety has 0-3 substituent(s) selected from F, Cl, Br, or I;
R₁₃ is —OR₁₄, —SR₁₄, —N(R₁₄)₂, —C(O)R₁₄, —C(O)N(R₁₄)₂, —CN, —CF₃, —NR₁₄C(O)R₁₄, —S(O)₂N(R₁₄)₂, —NR₁₄S(O)₂R₁₄, or —NO₂;
Each R₁₄ is independently H, alkyl, cycloalkyl, heterocycloalkyl, haloalkyl, halocycloalkyl, or haloheterocycloalkyl;
wherein W is (A): 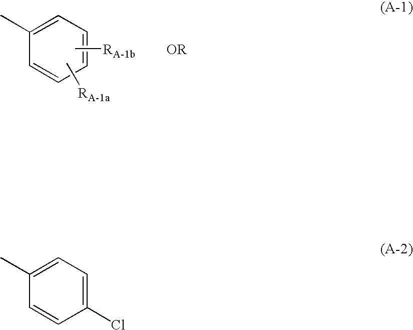
R_{A-1a} is H, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, aryl, —R₅, R₆, —OR_{A-3}, —OR_{A-4}, —SR_{A-3}, F, Cl, Br, I, —N(R_{A-3})₂, —N(R_{A-5})₂, —C(O)R_{A-3}, —C(O)R_{A-5}, —CN, —C(O)N(R_{A-3})₂, —C(O)N(R_{A-6})₂, —NR_{A-3}C(O)R_{A-3}, —S(O)R_{A-3}, —OS(O)₂R_{A-3}, —NR_{A-3}S(O)₂R_{A-3}, —NO₂, and —N(H)C(O)N(H)R_{A-3};
R_{A-1b} is —O-R_{A-3}, —S-R_{A-3}, —S(O)—R_{A-3}, —C(O)—R_{A-7}, and alkyl substituted on the o) carbon with R_{A-7;}
Each R_{A-3} is independently selected from H, alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;
R_{A-4} is selected from cycloalkyl, halocycloalkyl, substituted cycloalkyl, heterocycloalkyl, haloheterocycloalkyl, or substituted heterocycloalkyl;
Each R_{A-5} is independently selected from cycloalkyl, halocycloalkyl, substituted cycloalkyl, heterocycloalkyl, haloheterocycloalkyl, substituted heterocycloalkyl, R₅, R₆, phenyl, or substituted phenyl;
Each R_{A-6} is independently selected from alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;
R_{A-7} is selected from aryl, R₅, or R₆;
wherein W is (B): 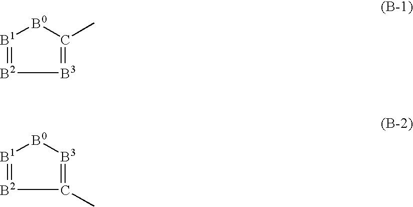
B° is —O—, —S—, or —N(R_{B-0})—;
B¹ and B² are independently selected from ═N—, or ═C(R_{B-1})—;
B³ is ═N—, or ═CH—, provided that when both B¹ and B² are ═C(R_{B-1})— and B³ is ═CH—, only one ═C(R_{B-1})— can be ═CH—, and further provided that when B⁰ is —O—, B² is ═C(RB-])- and B³ is ═C(H)—, B¹ cannot be═N—,
R_{B-0} is H, alkyl, cycloalkyl, heterocycloalkyl, haloalkyl, halocycloalkyl, haloheterocycloalkyl, substituted alkyl, limited substituted alkyl, substituted cycloalkyl, substituted heterocycloalkyl, or aryl, and provided that when B is (B-2) and B³ is ═N— and B⁰ is N(RB-o), R_{B-0} cannot be phenyl or substituted phenyl;
R_{B-1} is H, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, limited substituted alkyl, limited substituted alkenyl, limited substituted alkynyl, aryl, —OR_{B-2}, —OR_{B-3}, —SR_{B-2}, —SR_{B-3}, F, Cl, Br, I, —N(R_{B-2})₂, —N(R_{B-3})₂, —C(O)R_{B-2}, —C(O)R_{B-3}, —C(O)N(R_{B-2})₂, —C(O)N(R_{B-3})₂, —CN, —NR_{B-2}C(O)R_{B-4}, —S(O)₂N(R_{B-2})₂, —OS(O)₂R_{B-4}, —S(O)₂R_{B-2}, —S(O)₂R_{B-3}, —NR_{B-2}S(O)₂R_{B-2}, —N(H)C(O)N(H)R_{B-2}, —NO₂, R₅, and R₆;
Each R_{B-2} is independently H, alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;
Each R_{B-3} is independently H, alkyl, haloalkyl, limited substituted alkyl, cycloalkyl, halocycloalkyl, substituted cycloalkyl, heterocycloalkyl, haloheterocycloalkyl, substituted heterocycloalkyl;
R_{B-4} is independently H, alkyl, cycloalkyl, heterocycloalkyl, haloalkyl, halocycloalkyl, or haloheterocycloalkyl;
wherein W is (C):
(C) is a six-membered heterocyclic ring system having 1-2 nitrogen atoms or a 10-membered bicyclic-six-six-fused-ring system having up to two nitrogen atoms within either or both rings, provided that no nitrogen is at a bridge of the bicyclic-six-six-fused-ring system, and further having 1-2 substitutents independently selected from R_{C-1};
Each R_{C-1} is independently H, F, Cl, Br, I, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocyloalkyl, substituted heterocycloalkyl, lactam heterocycloalkyl, phenyl, substituted phenyl, —NO₂, —CN, —OR_{C-2}, —SR_{C-2}, —SOR_{C-2}, —SO₂R_{C-2}, —NR_{C-2}C(O)R_{C-3}, —NR_{C-2}C(O)R_{C-2}, —NR_{C-2}C(O)R_{C-4}, —N(R_{C-2})₂, —C(O)R_{C-2}, —C(O)₂R_{C-2}, —C(O)N(R_{C-2})₂, —SCN, —NR_{C-2}C(O)R_{C-2}, —S(O)N(R_{C-2})₂, —S(O)₂N(R_{C-2})₂, —NR_{C-2}S(O)₂R_{C-2}, R₅, or R₆;
Each R_{C-2} is independently H, alkyl, cycloalkyl, heterocycloalkyl, alkyl substituted with 1 substituent selected from R_{C-5}, cycloalkyl substituted with 1 substituent selected from R_{C-5}, heterocycloalkyl substituted with 1 substituent selected from R_{C-5}, haloalkyl, halocycloalkyl, haloheterocycloalkyl, phenyl, or substituted phenyl;
Each R_{C-3} is independently H, alkyl, or substituted alkyl;
R_{C-4} is H, alkyl, an amino protecting group, or an alkyl group having 1-3 substituents selected from F, Cl, Br, I, —OH, —CN, —NH₂, —NH(alkyl), or —N(alkyl)₂;
R_{C-5} is —CN, —CF₃, —NO₂, —OR_{C-6}, —SR_{C-6}, —N(R_{C-6})₂, —C(O)R_{C-6}, —SOR_{C-6}, —SO₂RR_{C-6}, —C(O)N(R_{C-6})₂, —NR_{C-6}C(O)R_{C-6}, —S(O)₂N(R_{C-6})₂, or —NR_{C-6}S(O)₂R_{C-6};
Each R_{C-6} is independently H, alkyl, cycloalkyl, heterocycloalkyl, haloalkyl, halocycloalkyl, or haloheterocycloalkyl;
wherein W is (D): 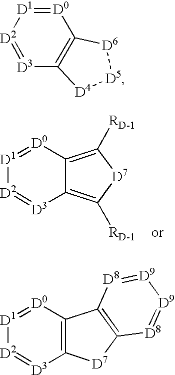
provided that the bond between the —C(═X)— group and the W group may be attached at any available carbon atom within the D group as provided in R_{D-1}, R_{D-3}, and R_{D-4;}
D⁰, D¹, D², and D³ are N or C(R_{D-1}) provided that up to one of D⁰, D¹, D², or D³ is N and the others are C(R_{D-1}), further provided that when C(X) is attached at D² and D⁰ or D¹ is N, D³ is C(H), and further provided that there is only one attachment to C(X);
D⁴—D⁵—D⁶ is selected from N(R_{D-2})—C(R_{D-3})═C(R_{D-3}), N═C(R_{D-3})—C(R_{D-4})₂, C(R_{D-3})═C(R_{D-3})—N(R_{D-2}), C(R_{D-3})₂—N(R_{D-2})—C(R_{D-3})₂, C(R_{D-4})₂—C(R_{D-3})═N, N(R_{D-2})—C(R_{D-3})₂—C(R_{D-3})₂, C(R_{D-3})₂—C(R_{D-3})₂—N(R_{D-2}), O—C(R_{D-3})═C(R_{D-3}), O—C(R_{D-3})₂—C(R_{D-3})₂, C(R_{D-3})₂—O—C(R_{D-3})₂, C(R_{D-3})═C(R_{D-3})—O, C(R_{D-3})₂—C(R_{D-3})₂—O, S—C(R_{D-3})═C(R_{D-3}), S—C(R_{D-3})₂—C(R_{D-3})₂, C(R_{D-3})₂—S—C(R_{D-3})₂, C(R_{D-3})═C(R_{D-3})—S, or C(R_{D-3})₂—C(R_{D-3})2_S;
provided that when C(X) is attached to W at D² and D⁶ is O, N(R_{D-2}), or S, D⁴—D⁵ is not CH═CH;
and further provided that when C(X) is attached to W at D² and D⁴ is O, N(R_{D-2}), or S, D⁵—D⁶ is not CH═CH;
Each R_{D-1} is independently H, F, Br, I, Cl, —CN, —CF₃, —OR_{D-5}, —SR_{D-5}, —N(R_{D-5})₂, or a bond to C(X) provided that only one R_{D-1} and no R_{D-3} or R_{D-4} is said bond,
Each R_{D-2} is independently H, alkyl, haloalkyl, substituted alkyl, cycloalkyl, halocycloalkyl, substituted cycloalkyl, heterocycloalkyl, haloheterocycloalkyl, substituted heterocycloalkyl, R₅, or R₆;
Each R_{D-3} is independently H, F, Br, Cl, I, alkyl, substituted alkyl, haloalkyl, alkenyl, substituted alkenyl, haloalkenyl, alkynyl, substituted alkynyl, haloalkynyl, heterocycloalkyl, substituted heterocycloalkyl, lactam heterocycloalkyl, —CN, —NO₂, —OR_{D-10}, —C(O)N(R_{D-11})₂, —NR_{D-10}COR_{D-12}, —N(R_{D-10})₂, —SR_{D-10}, —S(O)₂R_{D-10}, —C(O)R_{D-12}, —CO₂R_{D-10}, aryl, R₅, R₆, or a bond to C(X) provided that only one R_{D-3} and no R_{D-1} or R_{D-4} is also said bond;
Each R_{D-4} is independently H, F, Br, Cl, I, alkyl, substituted alkyl, haloalkyl, alkenyl, substituted alkenyl, haloalkenyl, alkynyl, substituted alkynyl, haloalkynyl, heterocycloalkyl, substituted heterocycloalkyl, lactam heterocycloalkyl, —CN, —NO₂, —OR_{D-10}, —C(O)N(R_{D-11})₂, —NR_{D-10}COR_{D-12}, —N(R_{D-11})₂, —SR_{D-10}, —CO₂R_{D-10}, aryl, R₅, R₆, or a bond to C(X) provided that only one R_{D-4} and no R_{D-1} or R_{D-3} is also said bond;
Each R_{D-5} is independently H, C₁₋₃ alkyl, or C₂₋₄ alkenyl;
D⁷ is O, S, or N(R_{D-2});
D⁸ and D⁹ are C(R_{D-1}), provided that when C(X) is attached at a D⁹, each D⁸ is CH;
Each R_{D-10} is H, alkyl, cycloalkyl, haloalkyl, substituted phenyl, or substituted naphthyl;
Each R_{D-11} is independently H, alkyl, cycloalkyl, heterocycloalkyl, alkyl substituted with 1 substituent selected from R₁₃, cycloalkyl substituted with 1 substituent selected from R₁₃, heterocycloalkyl substituted with 1 substituent selected from R₁₃, haloalkyl, halocycloalkyl, haloheterocycloalkyl, phenyl, or substituted phenyl;
R_{D-12} is H, alkyl, substituted alkyl, cycloalkyl, haloalkyl, heterocycloalkyl, substituted heterocycloalkyl, substituted phenyl, or substituted naphthyl;
wherein W is (E): 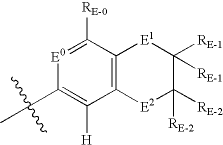
E⁰ is CH or N;
R_{E-0} is H, F, Cl, Br, I, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, aryl, R₅, R₆, —OR_{E-3}, —OR_{E-4}, —SR_{E-3}, —SR_{E-5}, —N(R_{E-3})₂, —NR_{E-3}R_{E-6}, —N(R_{E-6})₂, —C(O)R_{E-3}, —CN, —C(O)N(R_{E-3})₂, —NR_{E-3}C(O)R_{E-3}, —S(O)R_{E-3}, —S(O)R_{E-5}, —OS(O)₂R_{E-3}, —NR_{E-3}S(O)₂R_{E-3}, —NO₂, or —N(H)C(O)N(H)R_{E-3};
E¹ is O, CR_{E-1-1}, or C(R_{E-1-1})₂, provided that when El is CR_{E-1-1}, one R_{E-1} is a bond to CR_{E-1-1}, and further provided that at least one of E¹ or E² is O;
Each R_{E-1-1} is independently H, F, Br, Cl, CN, alkyl, haloalkyl, substituted alkyl, alkynyl, cycloalkyl, —OR_{E}, or —N(R_{E})₂, provided that at least one R_{E-1-1} is H when E¹ is C(R_{E-1-1})₂;
Each R_{E-1} is independently H, alkyl, substituted alkyl, haloalkyl, cycloalkyl, heterocycloalkyl, or a bond to E¹ provided that E¹ is CR_{E-1-1;}
E² is O, CR_{E-2-2}, or C(R_{E-2-2})₂, provided that when E² is CR_{E-2-2}, one R_{E-2} is a bond to CR_{E-2-2}, and further provided that at least one of E¹ or E is O;
Each R_{E-2-2} is independently H, F, Br, Cl, CN, alkyl, haloalkyl, substituted alkyl, alkynyl, cycloalkyl, —OR_{E}, or —N(R_{E})₂, provided that at least one R_{E-2-2} is H when E² is C(R_{E-2-2})₂;
Each R_{E-2} is independently H, alkyl, substituted alkyl, haloalkyl, cycloalkyl, heterocycloalkyl, or a bond to E provided that E² is CR_{E-2-2};
Each R_{E} is independently H, alkyl, cycloalkyl, heterocycloalkyl, haloalkyl, halocycloalkyl, or haloheterocycloalkyl;
Each R_{E-3} is independently H, alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or phenyl having 1 substituent selected from R₉ and further having 0-3 substituents independently selected from F, Cl, Br, or I or substituted phenyl;
R_{E-4} is H, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;
Each R_{E-5} is independently H, haloalkyl, substituted alkyl, cycloalkyl, halocycloalkyl, substituted cycloalkyl, heterocycloalkyl, haloheterocycloalkyl, substituted heterocycloalkyl, R₅, or R₆;
Each R_{E-6} is independently alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or phenyl having 1 substituent selected from R₉ and further having 0-3 substituents independently selected from F, Cl, Br, or I;
wherein W is (F): 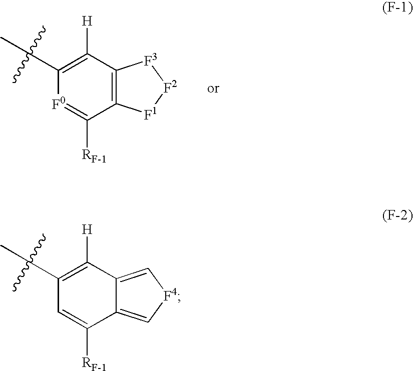
F° is C(H), wherein F¹—F²—F³ is selected from O—C(R_{F-2})═N, O—C(R_{F-3})(R_{F-2})—N(R_{F-4}), O—C(R_{F-3})(R_{F-2})—S, O—N═C(R_{F-3}), O—C(R_{F-2})(R_{F-3})—O, S—C(R_{F-2})═N, S—C(R_{F-3})(R_{F-2})—N(R_{F-4}), S—N═C(R_{F-3}), N═C(R_{F-2})—O, N═C(R_{F-2})—S, N═C(R_{F-2})—N(R_{F-4}), N(R_{F-4})—N═C(R_{F-3}), N(R_{F-4})—C(R_{F-3})(R_{F-2})—O, N(R_{F-4})—C(R_{F-3})(R_{F-2})—S, N(R_{F-4})—C(R_{F-3})(R_{F-2})—N(R_{F-4}), C(R_{F-3})₂—O—N(R_{F-4}), C(R_{F-3})₂—N(R_{F-4})—O, C(R_{F-3})₂—N(R_{F-4})—S, C(R_{F-3})═N—O, C(R_{F-3})═N—S, C(R_{F-3})═N—N(R_{F-4}), or C(R_{F-3})₂—C(R_{F-2})(R_{F-3})—C(R_{F-3})₂; or
F⁰ is N, wherein F¹—F²—F³ is selected from O—C(R_{F-2})═N, O—C(R_{F-3})(R_{F-2})—N(R_{F-4}), O—C(R_{F-3})(R_{F-2})—S, O—N═C(R_{F-3}) O—C(R_{F-2})(R_{F-3})—O, S—C(R_{F-2})═N, S—C(R_{F-3})(R_{F-2})—N(R_{F-4}), S—N═C(R_{F-3}), N═C(R_{F-2})—O, N═C(R_{F-2})—S, N═C(R_{F-2})—N(R_{F-4}), N(R_{F-4})—N═C(R_{F-3}), N(R_{F-4})—C(R_{F-3})(R_{F-2})—O, N(R_{F-4})—C(R_{F-3})(R_{F-2})—S, N(R_{F-4})—C(R_{F-3})(R_{F-2})—N(R_{F-4}), C(R_{F-3})₂—O—N(R_{F-4}), C(R_{F-3})₂—N(R_{F-4})—O, C(R_{F-3})₂—N(R_{F-4})—S, C(R_{F-3})═N—O, C(R_{F-3})═N—S, C(R_{F-3})═N—N(R_{F-4}), C(R_{F-3})=C(R_{F-2})—C(R_{F-3})₂, or C(R_{F-3})₂—C(R_{F-2})(R_{F-3})—C(R_{F-3})₂;
F⁴ is N(R_{F-7}), O, or S;
R_{F-1} is H, F, Cl, Br, I, —CN, —CF₃, —OR_{F-8}, —SR_{F-8}, or —N(R_{F-8})₂;
R_{F-2} is H, F, alkyl, haloalkyl, substituted alkyl, lactam heterocycloalkyl, phenoxy, substituted phenoxy, R₅, R₆, —N(R_{F-4})-aryl, —N(R_{F-4})-substituted phenyl, —N(R_{F-4})-substituted naphthyl, —O-substituted phenyl, —O-substituted naphthyl, —S-substituted phenyl, —S-substituted naphthyl, or alkyl substituted on the c carbon with R_{F-9};
R_{F-3} is H, F, Br, Cl, I, alkyl, substituted alkyl, haloalkyl, alkenyl, substituted alkenyl, haloalkenyl, alkynyl, substituted alkynyl, haloalkynyl, heterocycloalkyl, substituted heterocycloalkyl, lactam heterocycloalkyl, —CN, —NO₂, —OR_{F-8}, —C(O)N(R_{F-8})₂, —NHR_{F-8}, —NR_{F-8}COR_{F-8}, —N(R_{F-8})₂, —SR_{F-8}, —C(O)R_{F-8}, —CO₂R_{F-8}, aryl, R₅, or R₆;
R_{F-4} is H, or alkyl;
R_{F-7} is H, alkyl, haloalkyl, substituted alkyl, cycloalkyl, halocycloalkyl, substituted cycloalkyl, phenyl, or phenyl having 1 substituent selected from R₉ and further having 0-3 substituents independently selected from F, Cl, Br, or I;
R_{F-8} is H, alkyl, substituted alkyl, cycloalkyl, haloalkyl, heterocycloalkyl, substituted heterocycloalkyl, substituted phenyl, or substituted naphthyl;
R_{F-9} is aryl, R₅, or R₆;
wherein W is (G): 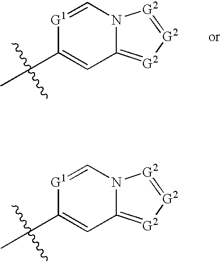
G¹ is N or CH;
Each G² is N or C(R_{G-1}), provided that no more than one G is N, and further provided that when G² adjacent to the bridge N is C(R_{G-1}) and the other G² are CH, that R_{G-1} is other than H, F, Cl, I, alkyl, substituted alkyl or alkynyl;
Each R_{G-1} is independently H, alkyl, substituted alkyl, haloalkyl, alkenyl, substituted alkenyl, haloalkenyl, alkynyl, substituted alkynyl, haloalkynyl, —CN, —NO₂, F, Br, Cl, I, —C(O)N(R_{G-3})₂, —N(R_{G-3})₂, —SR_{G-6}, —S(O)₂R_{G-6}, —OR_{G-6}, —C(O)R_{G-6}, —CO₂R_{G-6}, aryl, R₅, R₆, or two R_{G-1} on adjacent carbon atoms may combine for W to be a 6-5-6 fused-tricyclic-heteroaromatic-ring system optionally substituted on the newly formed ring where valency allows with 1-2 substitutents independently selected from F, Cl, Br, I, and R_{G-2;}
R_{G-2} is alkyl, alkenyl, alkynyl, cycloalkyl, heterocycloalkyl, haloalkyl, haloalkenyl, haloalkynyl, halocycloalkyl, haloheterocycloalkyl, —OR_{G-8}, —SR_{G-8}, —S(O)₂R_{G-8}, —S(O)R_{G-8}, —OS(O)₂R_{G-8}, —N(R_{G-8})₂, —C(O)R_{G-8}, —C(S)R_{G-8}, —C(O)OR_{G-8}, —CN, —C(O)N(R_{G-8})₂, —NR_{G-8}C(O)R_{G-8}, —S(O)₂N(R_{G-8})₂, —NR_{G-8}S(O)₂R_{G-8}, —NO₂, —N(R_{G-8})C(O)N(R_{G-8})₂, substituted alkyl, substituted alkenyl, substituted alkynyl, substituted cycloalkyl, substituted heterocycloalkyl, lactam heterocycloalkyl, phenyl, phenyl having 0-4 substituents independently selected from F, Cl, Br, I and R_{G-7}, naphthyl, or naphthyl having 0-4 substituents independently selected from F, Cl, Br, I, or R_{G-7;}
Each R_{G-3} is independently H, alkyl, cycloalkyl, heterocycloalkyl, alkyl substituted with 1 substituent selected from R_{G-4}, cycloalkyl substituted with I substituent selected from R_{G-4}, heterocycloalkyl substituted with 1 substituent selected from R_{G-4}, haloalkyl, halocycloalkyl, haloheterocycloalkyl, phenyl, or substituted phenyl;
R_{G-4} is —OR_{G-5}, —SR_{G-5}, —N(R_{G-5})₂, —C(O)R_{G-5}, —SOR_{G-5}, —SO₂R_{G-5}, —C(O)N(R_{G-5})₂, —CN, —CF₃, —NR_{G-5}C(O)R_{G-5}, —S(O)₂N(R_{G-5})₂, —NR_{G-5}S(O)₂R_{G-5}, or —NO₂;
Each R_{G-5} is independently H, alkyl, cycloalkyl, heterocycloalkyl, haloalkyl, halocycloalkyl, or haloheterocycloalkyl;
R_{G-6} is H, alkyl, haloalkyl, substituted alkyl, cycloalkyl, halocycloalkyl, substituted cycloalkyl, phenyl, or phenyl having 0-4 substituents independently selected from F, Cl, Br, I, and R_{G-7;}
R_{G-7} is alkyl, substituted alkyl, haloalkyl, —OR_{G-5}, —CN, —NO₂, —N(R_{G-3})₂;
Each R_{G-8} is independently H, alkyl, haloalkyl, substituted alkyl, cycloalkyl, halocycloalkyl, substituted cycloalkyl, heterocycloalkyl, haloheterocycloalkyl, substituted heterocycloalkyl, phenyl, or phenyl substituted with 0-4 independently selected from F, Cl, Br, I, or R_{G-7;}
wherein W is (H) 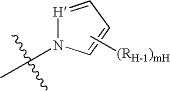
H¹ is N or CH;
Each R_{H-1}, is independently F, Cl, Br, I, —CN, —NO₂, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocyloalkyl, substituted heterocycloalkyl, lactam heterocyclcoalkyl, aryl, R₅, R₆, —OR_{H-3}, —SR_{H-3}, —SOR_{H-3}, —SO₂R_{H-3}, —SCN, —S(O)N(R_{H-3})₂, —S(O)₂N(R_{H-3})₂, —C(O)R_{H-3}, —C(O)₂R_{H-3}, —C(O)N(R_{H-3})₂, —C(R_{H-3})═N—OR_{H-3}, —NC(O)R_{H-3}, —NC(O)R_{H-3}, —NC(O)R_{H-3}, —N(R_{H-3})₂, —NR_{H-3}C(O)R_{H-3}, —NR_{H-3}S(O)₂R_{H-3}, or two R_{H-1} on adjacent carbon atoms may fuse to form a 6-membered ring to give a 5-6 fused, bicyclic moiety where the 6-membered ring is optionally substituted with 1-3 substitutents selected from R_{H-2};
m_{H} is 0, 1, or 2;
R_{H-2} is alkyl, alkenyl, alkynyl, cycloalkyl, heterocycloalkyl, haloalkyl, haloalkenyl, haloalkynyl, halocycloalkyl, haloheterocycloalkyl, —OR_{H-3}, —SR_{H-3}, —S(O)₂R_{H-3}, —S(O)R_{H-3}, —OS(O)₂R_{H-3}, —N(R_{H-3})₂, —C(O)R_{H-3}, —C(S)R_{H-3}, —C(O)OR_{H-3}, —CN, —C(O)N(R_{H-3})₂, —NR_{H-3}C(O)R_{H-3}, —S(O)₂N(R_{H-3})₂, —NR_{H-3}S(O)₂R_{H-3}, —NO₂, —N(R_{H-3})C(O)N(R_{H-3})₂, substituted alkyl, substituted alkenyl, substituted alkynyl, substituted cycloalkyl, substituted heterocycloalkyl, lactam heterocycloalkyl, phenyl, phenyl having 0-4 substituents independently selected from F, Cl, Br, I and R₇, naphthyl, naphthyl having 0-4 substituents independently selected from F, Cl, Br, I, or R₇, or two R_{H-2} on adjacent carbon atoms may combine to form a three-ring-fused-5-6-6 system optionally substituted with up to 3 substituents independently selected from Br, Cl, F, I, —CN, —NO₂, —CF₃, —N(R_{H-3})₂, —N(R_{H-3})C(O)R_{H-3}, alkyl, alkenyl, and alkynyl;
Each R_{H-3} is independently H, alkyl, haloalkyl, substituted alkyl, cycloalkyl, halocycloalkyl, substituted cycloalkyl, heterocycloalkyl, haloheterocycloalkyl, substituted heterocycloalkyl, phenyl, or phenyl substituted with 0-4 independently selected from F, Cl, Br, I, or R₇;
pharmaceutically acceptable salt, racemic mixture, or pure enantiomer thereof; and provided that the compound of Formula I includes at least one isotopic label.

2. The compound of claim 1, wherein R₂ is H or CH₃, each R₃ is H, and R₄ is H.

3. The compound of claim 2, wherein W is 4-chlorobenz-1-yl; dibenzo[b,d]thiophene-2-yl; isoquinoline-3-yl; furo[2,3-c]pyridine-5-yl; 1,3-benzodioxole-5-yl; 2,3-dihydro-1,4-benzodioxine-6-yl; 1,3-benzoxazole-5-yl; thieno[2,3-c]pyridine-5-yl; thieno[3,2-c]pyridine-6-yl; [I]benzothieno[3,2-c]pyridine-3-yl; 1,3-benzothiazole-6-yl; thieno[3,4-c]pyridine-6-yl; 2,3-dihydro-1-benzofuran-5-yl; 1-benzofuran-5-yl; furo[3,2-c]pyridine-6-yl; [1]benzothieno[2,3-c]pyridine-3-yl; dibenzo[b,d]furan-2-yl; 1-benzofuran-6-yl; 2-naphthyl; 1H-indole-6-yl; pyrrolo[1,2-c]pyrimidine-3-yl; 1-benzothiophene-5-yl; 1-benzothiophene-5-yl; 1-benzothiophene-6-yl; pyrrolo[1,2-a]pyrazine-3-yl; 1H-indole-6-yl; pyrazino[1,2-a]indole-3-yl; 1,3-benzothiazole-6-yl; [1]benzofuro[2,3-c]pyridine-3-yl; [1]benzofuro[2,3-c]pyridine-3-yl; 2H-chromene-6-yl; indolizine-6-yl; and [1,3]dioxolo[4,5-c]pyridine-6-yl; any of which is optionally substituted as allowed in claim 1.

4. The compound of claim 2, wherein W is thiophene-2-yl, furan-2-yl, pyrrole-2-yl, 1,3-oxazole-2-yl, 1,3-thiazole-2-yl, isoxazole-3-yl, isothiazole-3-yl; any of which is optionally substituted at the 5 position on the ring as allowed in formula I, and 1,3-oxazole-4-yl, 1,3-oxazole-5-yl, 1,3-thiazole-4-yl, 1,3-thiazole-5-yl; any of which is optionally substituted at the 2 position on the ring as allowed in claim 1.

5. The compound of claim 2, wherein the compound is:
N-[(3R)-1-azabicyclo[2.2.2]oct-3-yl]-1-benzofuran-5-[¹¹C]carboxamide;
N-[(2S,3R)-2-methyl-1-azabicyclo[2.2.2]oct-3-yl]-1-benzofuran-5-[¹¹C]carboxamide;
N-[(3R,5R)-1-azabicyclo[3.2.1]oct-3-yl]-1-benzofuran-5-[¹¹C]carboxamide;
N-[(3R)-1-azabicyclo[2.2.2]oct-3-yl]-2-methyl-1-benzofuran-5-[¹¹C]carboxamide;
N-[(2S,3R)-2-methyl-1-azabicyclo[2.2.2]oct-3-yl]-2-methyl-1-benzofuran-5-[¹¹C]carboxamide;
N-[(3R,5R)-1-azabicyclo[3.2.1]oct-3-yl]-2-methyl-1-benzofuran-5-[¹¹C]carboxamide;
N-[(3R)-1-azabicyclo[2.2.2]oct-3-yl]furo[2,3-c]pyridine-5-[¹¹C]carboxamide;
N-[(2S,3R)-2-methyl-1-azabicyclo[2.2.2]oct-3-yl]furo[2,3-c]pyridine-5-[¹¹C]carboxamide;
N-[(2R)-7-azabicyclo[2.2.1]hept-2-yl]furo[2,3-c]pyridine-5-[¹¹C]carboxamide;
N-[(3R,5R)-1-azabicyclo[3.2.1]oct-3-yl]furo[2,3-c]pyridine-5-[¹¹C]carboxamide;
N-[(3S)-1-azabicyclo[2.2.2]oct-3-yl]-3-methylfuro[2,3-c]pyridine-5-[¹¹C]carboxamide;
N-[(3R)-1-azabicyclo[2.2.2]oct-3-yl]-5-bromothiophene-2-[¹¹C]carboxamide;
5-bromo-N-[(2S,3R)-2-methyl-1-azabicyclo[2.2.2]oct-3-yl]thiophene-2-[¹¹C]carboxamide;
N-[(3R)-1-azabicyclo[2.2.2]oct-3-yl]-5-pyridin-2-ylthiophene-2-[¹¹C]carboxamide;
N-[(2S,3R)-2-methyl-1-azabicyclo[2.2.2]oct-3-yl]-5-pyridin-2-ylthiophene-2-[¹¹C]carboxamide;
N-[(3R)-1-azabicyclo[2.2.2]oct-3-yl]-5-(methylthio)thiophene-2-[¹¹C]carboxamide;
N-[(2S,3R)-2-methyl-1-azabicyclo[2.2.2]oct-3-yl]-5-(methylthio)thiophene-2-[¹¹C]carboxamide;
N-[(3R)-1-azabicyclo[2.2.2]oct-3-yl]-5-phenylthiophene-2-[¹¹C]carboxamide;
N-[(3R)-1-azabicyclo[2.2.2]oct-3-yl]-5-methoxythiophene-2-[¹¹C]carboxamide;
N-[(2S,3R)-2-methyl-1-azabicyclo[2.2.2]oct-3-yl]-5-methoxythiophene-2-[¹¹C]carboxamide;
N-[(3R)-1-azabicyclo[2.2.2]oct-3-yl]-5-nitrothiophene-2-[¹¹C]carboxamide;
N-[(2S,3R)-2-methyl-1-azabicyclo[2.2.2]oct-3-yl]-5-nitrothiophene-2-[¹¹C]carboxamide;
N-[(3R)-1-azabicyclo[2.2.2]oct-3-yl]-5-(2-[⁸F]fluorophenyl)-2-furamide;
5-(2-[1⁸F]fluorophenyl)-N-[(2S,3R)-2-methyl-1-azabicyclo[2.2.2]oct-3-yl]-2-furamide;
N-[(3R)-1-azabicyclo[2.2.2]oct-3-yl]-4-[¹²³I]iodo-1H-pyrazole-1-carboxamide;
N-[(2S,3R)-2-methyl-1-azabicyclo[2.2.2]oct-3-yl]-4-[123I]iodo-1H-pyrazole-1-carboxamide;
N-[(3R,5R)-1-azabicyclo[3.2. I]oct-3-yl]-4-[¹²³]iodo-1H-pyrazole-1-carboxamide; or pharmaceutically acceptable salts thereof.

6. The compound of claim 2, wherein the compound is
N-[(3R)-1-azabicyclo[2.2.2]oct-3-yl]-1-benzofuran-5-[¹³C]carboxamide;
N-[(2S,3R)-2-methyl-1-azabicyclo[2.2.2]oct-3-yl]-1-benzofuran-5-[¹³C]carboxamide;
N-[(3R,5R)-1-azabicyclo[3.2.1]oct-3-yl]-1-benzofuran-5-[¹³C]carboxamide;
N-[(3R)-1-azabicyclo[2.2.2]oct-3-yl]-2-methyl-1-benzofuran-5-[¹³C]carboxamide;
N-[(2S,3R)-2-methyl-1-azabicyclo[2.2.2]oct-3-yl]-2-methyl-1-benzofuran-5-[¹³C]carboxamide;
N-[(3R,5R)-1-azabicyclo[3.2.1]oct-3-yl]-2-methyl-1-benzofuran-5-[¹³C]carboxamide;
N-[(3R)-1-azabicyclo[2.2.2]oct-3-yl]furo[2,3-c]pyridine-5-[¹³C]carboxamide;
N-[(2S,3R)-2-methyl-1-azabicyclo[2.2.2]oct-3-yl]furo[2,3-c]pyridine-5-[¹³C]carboxamide;
N-[(2R)-7-azabicyclo[2.2.1]hept-2-yl]furo[2,3-c]pyridine-5-[¹³C]carboxamide;
N-[(3R,5R)-1-azabicyclo[3.2.1]oct-3-yl]furo[2,3-c]pyridine-5-[¹³C]carboxamide;
N-[(3S)-1-azabicyclo[2.2.2]oct-3-yl]-3-methylfuro[2,3-c]pyridine-5-[¹³C]carboxamide;
N-[(3R)-1-azabicyclo[2.2.2]oct-3-yl]-5-bromothiophene-2-[¹³C]carboxamide;
5-bromo-N-[(2S,3R)-2-methyl-1-azabicyclo[2.2.2]oct-3-yl]thiophene-2-[13C]carboxamide;
N-[(3R)-1-azabicyclo[2.2.2]oct-3-yl]-5-pyridin-2-ylthiophene-2-[¹³C]carboxamide;
N-[(2S,3R)-2-methyl-1-azabicyclo[2.2.2]oct-3-yl]-5-pyridin-2-ylthiophene-2-[¹³C]carboxamide;
N-[(3R)-1-azabicyclo[2.2.2]oct-3-yl]-5-(methylthio)thiophene-2-[¹³C]carboxamide;
N-[(2S,3R)-2-methyl-1-azabicyclo[2.2.2]oct-3-yl]-5-(methylthio)thiophene-2-[¹³C]carboxamide;
N-[(3R)-1-azabicyclo[2.2.2]oct-3-yl]-5-phenylthiophene-2-[¹³C]carboxamide;
N-[(3R)-1-azabicyclo[2.2.2]oct-3-yl]-5-methoxythiophene-2-[¹³C]carboxamide;
N-[(2S,3R)-2-methyl-1-azabicyclo[2.2.2]oct-3-yl]-5-methoxythiophene-2-[¹³C]carboxamide;
N-[(3R)-1-azabicyclo[2.2.2]oct-3-yl]-5-nitrothiophene-2-[1³C]carboxamide;
N-[(2S,3R)-2-methyl-1-azabicyclo[2.2.2]oct-3-yl]-5-nitrothiophene-2-[¹³C]carboxamide;
N-[(3R)-1-azabicyclo[2.2.2]oct-3-yl]-5-(2-[¹⁹F]fluorophenyl)-2-furamide;
5-(2-[¹⁹F]fluorophenyl)-N-[(2S,3R)-2-methyl-1-azabicyclo[2.2.2]oct-3-yl]-2-furamide; or pharmaceutically acceptable salts thereof

7. A method for imaging and quantifying a compound of claim 1 in a subject, wherein said compound in the subject is indicative of the presence of selective nAChR in the subject.

8. The method of claim 7, wherein the compound is detected using position emission topography.

9. The method of claim 8, wherein the compound is
N-[(3R)-1-azabicyclo[2.2.2]oct-3-yl]-1-benzofuran-5-[¹¹C]carboxamide;
N-[(2S,3R)-2-methyl-1-azabicyclo[2.2.2]oct-3-yl]-1-benzofuran-5-[¹¹C]carboxamide;
N-[(3R,5R)-1-azabicyclo[3.2.1]oct-3-yl]-1-benzofuran-5-[¹¹C]carboxamide;
N-[(3R)-1-azabicyclo[2.2.2]oct-3-yl]-2-methyl-1-benzofuran-5-[¹¹C]carboxamide;
N-[(2S,3R)-2-methyl-1-azabicyclo[2.2.2]oct-3-yl]-2-methyl-1-benzofuran-5-[¹¹C]carboxamide;
N-[(3R,5R)-1-azabicyclo[3.2.1]oct-3-yl]-2-methyl-1-benzofuran-5-[¹¹C]carboxamide;
N-[(3R)-1-azabicyclo[2.2.2]oct-3-yl]furo[2,3-c]pyridine-5-[¹¹C]carboxamide;
N-[(2S,3R)-2-methyl-1-azabicyclo[2.2.2]oct-3-yl]furo[2,3-c]pyridine-5-[¹¹C]carboxamide;
N-[(2R)-7-azabicyclo[2.2.1]hept-2-yl]furo[2,3-c]pyridine-5-[¹¹C]carboxamide;
N-[(3R,5R)-1-azabicyclo[3.2.1]oct-3-yl]furo[2,3-c]pyridine-5-[¹¹C]carboxamide;
N-[(3S)-1-azabicyclo[2.2.2]oct-3-yl]-3-methylfuro[2,3-c]pyridine-5-[¹¹C]carboxamide;
N-[(3R)-1-azabicyclo[2.2.2]oct-3-yl]-5-bromothiophene-2-[¹¹C]carboxamide;
5-bromo-N-[(2S,3R)-2-methyl-1-azabicyclo[2.2.2]oct-3-yl]thiophene-2-[¹¹C]carboxamide;
N-[(3R)-1-azabicyclo[2.2.2]oct-3-yl]-5-pyridin-2-ylthiophene-2-[¹¹C]carboxamide;
N-[(2S,3R)-2-methyl-1-azabicyclo[2.2.2]oct-3-yl]-5-pyridin-2-ylthiophene-2-[¹¹C]carboxamide;
N-[(3R)-1-azabicyclo[2.2.2]oct-3-yl]-5-(methylthio)thiophene-2-[¹¹C]carboxamide;
N-[(2S,3R)-2-methyl-1-azabicyclo[2.2.2]oct-3-yl]-5-(methylthio)thiophene-2-[¹¹C]carboxamide;
N-[(3R)-1-azabicyclo[2.2.2]oct-3-yl]-5-phenylthiophene-2-[¹¹C]carboxamide;
N-[(3R)-1-azabicyclo[2.2.2]oct-3-yl]-5-methoxythiophene-2-[¹¹C]carboxamide;
N-[(2S,3R)-2-methyl-1-azabicyclo[2.2.2]oct-3-yl]-5-methoxythiophene-2-[¹¹C]carboxamide;
N-[(3R)-1-azabicyclo[2.2.2]oct-3-yl]-5-nitrothiophene-2-[¹¹C]carboxamide;
N-[(2S,3R)-2-methyl-1-azabicyclo[2.2.2]oct-3-yl]-5-nitrothiophene-2-[¹¹C]carboxamide;
N-[(3R)-1-azabicyclo[2.2.2]oct-3-yl]-5-(2-[¹⁸F]fluorophenyl)-2-furamide;
5-(2-[¹⁸F]fluorophenyl)-N-[(2S,3R)-2-methyl-1-azabicyclo[2.2.2]oct-3-yl]-2-furamide; or pharmaceutically acceptable salts thereof.

10. The method of claim 8, wherein the compound is
N-[(3R)-1-azabicyclo[2.2.2]oct-3-yl]-1-benzofuran-5-[¹¹C]carboxamide;
N-[(2S,3R)-2-methyl-1-azabicyclo[2.2.2]oct-3-yl]-1-benzofuran-5-[¹¹C]carboxamide;
N-[(3R,5R)-1-azabicyclo[3.2.1]oct-3-yl]-1-benzofuran-5-[¹¹C]carboxamide;
N-[(3R)-1-azabicyclo[2.2.2]oct-3-yl]-2-methyl-1-benzofuran-5-[¹¹C]carboxamide;
N-[(2S,3R)-2-methyl-1-azabicyclo[2.2.2]oct-3-yl]-2-methyl-1-benzofuran-5-[¹¹C]carboxamide;
N-[(3R,5R)-1-azabicyclo[3.2. I]oct-3-yl]-2-methyl-1-benzofuran-5-[¹¹C]carboxamide;
N-[(3R)-1-azabicyclo[2.2.2]oct-3-yl]furo[2,3-c]pyridine-5-[¹¹C]carboxamide;
N-[(2S,3R)-2-methyl-1-azabicyclo[2.2.2]oct-3-yl]furo[2,3-c]pyridine-5-[¹¹C]carboxamide;
N-[(2R)-7-azabicyclo[2.2.1]hept-2-yl]furo[2,3-c]pyridine-5-[¹¹C]carboxamide;
N-[(3R,5R)-1-azabicyclo[3.2.1]oct-3-yl]furo[2,3-c]pyridine-5-[¹¹C]carboxamide;
N-[(3S)-1-azabicyclo[2.2.2]oct-3-yl]-3-methylfuro[2,3-c]pyridine-5-[¹¹C]carboxamide; or pharmaceutically acceptable salts thereof.

11. The method of claim 7, wherein the compound is detected using single-photon emission computed tomography.

12. The method of claim 11, wherein the compound is
N-[(3R)-1-azabicyclo[2.2.2]oct-3-yl]-4-[¹²³I]iodo-1H-pyrazole-1-carboxamide;
N-[(2S,3R)-2-methyl-1-azabicyclo[2.2.2]oct-3-yl]-4-[¹²³I]iodo-1H-pyrazole-1-carboxamide;
N-[(3R,5R)-1-azabicyclo[3.2.1]oct-3-yl]-4-[123I]iodo-1H-pyrazole-1-carboxamide; or pharmaceutically acceptable salts thereof.

13. The method of claims 7, wherein the subject is a human patient.

14. The method of claim 7, wherein the detectably labeled compound comprises a moiety selected from the group consisting of ¹¹C, ¹⁸F, ⁷⁶Br, ¹²³I and ¹²⁵I.

15. The method of claims 7, wherein the disease is Alzheimer's disease, neurodegeneration associated with diseases such as Alzheimer's disease, pre-senile dementia (mild cognitive impairment), senile dementia, Parkinson's disease or schizophrenia.

16. The method of claim 7, wherein the disease is psychosis, attention deficit disorder, attention deficit hyperactivity disorder, depression, anxiety, general anxiety disorder, post traumatic stress disorder, or mood and affective disorders.

17. The method of claim 7, wherein the disease is amyotrophic lateral sclerosis, borderline personality disorder, traumatic brain injury, or behavioral and cognitive problems in general and associated with brain tumors.

18. The method of claim 7, wherein the disease is AIDS dementia complex, dementia associated with Down's syndrome, dementia associated with Lewy Bodies, Huntington's disease, tardive dyskinesia, Pick's disease, dysregulation of food intake including bulemia and anorexia nervosa, withdrawal symptoms associated with smoking cessation and dependant drug cessation, Gilles de la Tourette's Syndrome, age-related macular degeneration, glaucoma, neurodegeneration associated with glaucoma, diabetic retinopathy, or symptoms associated with pain.
